(19) United States
(12) Patent Application Publication
(10) Pub. No.: US 2014/0017260 A1
(43) Pub. Date: Jan. 16, 2014

(54) METHODS AND MATERIALS FOR REDUCING BONE LOSS

(71) Applicant: Mayo Foundation for Medical Education and Research, Rochester, MN (US)

(72) Inventors: David G. Monroe, Eyota, MN (US); Sundeep Khosla, Rochester, MN (US); Matthew M. Roforth, Plainview, MN (US)

(21) Appl. No.: 13/943,402

(22) Filed: Jul. 16, 2013

Related U.S. Application Data
(60) Provisional application No. 61/671,917, filed on Jul. 16, 2012.

(51) Int. Cl. A61K 39/395; C12N 15/113

(52) U.S. Cl. CPC A61K 39/3955; C12N 15/1138
USPC 424/172.1; 514/44 A

(57) ABSTRACT

This document provides methods and materials involved in reducing bone loss. For example, methods and material for using one or more inhibitors of a Rorβ polypeptide to reduce bone loss are provided. In some cases, methods and material for using one or more inhibitors of a Rorβ polypeptide to treat osteoporosis are provided.

### CROSS REFERENCE TO RELATED APPLICATIONS

This application claims the benefit of U.S. Provisional Application Ser. No. 61/671,917, filed Jul. 16, 2012. The disclosure of the prior application is considered part of (and is incorporated by reference in) the disclosure of this application.

### STATEMENT AS TO FEDERALLY SPONSORED RESEARCH

This invention was made with government support under AG004875 awarded by National Institutes of Health. The government has certain rights in the invention.

### BACKGROUND

1. Technical Field

This document relates to methods and materials involved in reducing bone loss. For example, this document provides methods and material for using one or more inhibitors of a retinoic acid receptor-related orphan receptor beta (Rorβ) polypeptide to reduce bone loss. In some cases, one or more inhibitors of a Rorβ polypeptide can be used as described herein to treat osteoporosis.

2. Background Information

Osteoporosis is a major health problem afflicting millions of people worldwide. It is most prevalent in postmenopausal women, but also occurs in a significant portion of men over the age of 50. In patients on glucocorticoids, and those undergoing hormone ablation therapy for either prostate or breast cancer, bone loss and osteoporosis are especially significant. In osteoporosis patients, the decrease of bone mineral density (BMD) and bone mass content (BMC) can result in increased bone fragility and increase risk of bone fracture. There are a number of drugs available for osteoporosis that prevent further bone loss (anti-resorptive agents), but the only FDA-approved anabolic (formation-stimulating) treatment for osteoporosis is teriparatide (also known as Forteo, Parathyroid Hormone (PTH)). While teriparatide can be initially effective, it may need to be given by injection, and its effects on increasing bone mass may wane after about 12-18 months.

### SUMMARY

This document provides methods and materials related to reducing bone loss. For example, this document provides methods and material for using one or more inhibitors of a Rorβ polypeptide to reduce bone loss. In some cases, one or more inhibitors of a Rorβ polypeptide can be used as described herein to treat osteoporosis.

As described herein, Rorβ polypeptide expression inhibits mineralization and expression of osteocalcin and osterix. In addition, suppression of Rorβ polypeptide expression results in enhanced expression of osterix. These results indicated that compositions containing one or more agents having the ability to inhibit Rorβ mRNA expression, Rorβ polypeptide expression, or Rorβ polypeptide activity can be used to reduce bone loss within a mammal (e.g., a human). Having the ability to reduce bone loss within a mammal can allow clinicians and patients to better treat and manage bone loss conditions such as osteoporosis.

In general, one aspect of this document features a method for reducing bone loss within a mammal. The method comprises, or consists essentially of, administering, to the mammal, an inhibitor of a Rorβ polypeptide under conditions wherein the rate of bone loss within the mammal is reduced. The mammal can be a human. The administration can be an oral or intravenous The rate of bone loss can be reduced by at least 50 percent.

In another aspect, this document features a method for reducing bone loss within a mammal. The method comprises, or consists essentially of, administering, to the mammal, a composition under conditions wherein the rate of bone loss within the mammal is reduced, wherein the composition comprises the ability to reduce Rorβ mRNA expression or Rorβ polypeptide expression. The mammal can be a human. The administration can be an oral or intravenous administration. The composition can comprise a nucleic acid construct having the ability to express a shRNA directed against Rorβ nucleic acid. The rate of bone loss can be reduced by at least 50 percent.

In another aspect, this document features a method for treating osteoporosis. The method comprises, or consists essentially of, administering, to a mammal having osteoporosis, an inhibitor of a Rorβ polypeptide under conditions wherein the rate of bone loss within the mammal is reduced or the bone mass within the mammal is increased. The mammal can be a human. The administration can be an oral or intravenous administration. The inhibitor can be an inhibitory anti-Rorβ polypeptide antibody. The rate of bone loss can be reduced by at least 50 percent or the bone mass within the mammal is increased by 15 percent.

In another aspect, this document features a method for treating osteoporosis. The method comprises, or consists essentially of, administering, to a mammal having osteoporosis, a composition under conditions wherein the rate of bone loss within the mammal is reduced or the bone mass within the mammal is increased, wherein the composition comprises the ability to reduce Rorβ mRNA expression or Rorβ polypeptide expression. The mammal can be a human. The administration can be an oral or intravenous administration. The composition can comprise a nucleic acid construct having the ability to express a shRNA directed against Rorβ nucleic acid. The rate of bone loss can be reduced by at least 50 percent or the bone mass within the mammal is increased by 15 percent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is contains photographs of Alizarin red-stained primary mouse calvarial osteoblasts at the indicated time points, depicting mineralization. FIGS. 1B, 1C, and 1D are graphs plotting relative expression of genes involved in forming the extracellular bone matrix (FIG. 1B), osteoblastic transcriptional regulation (FIG. 1C), and osteocyte biology (FIG. 1D) as determined by QPCR analysis at the indicated time points. The bars represent fold-induction relative to the expression at day 0 for each gene. The data are presented as the mean±SE and an asterisk (*) represents statistical significance of p≦0.01 (Student's t-test).

FIG. 2A contains a photograph of a heatmap generated from by the expression patterns of nuclear receptor genes at either 2 or 24 hours following osteoblastic induction. QPCR was performed on the 49 members of the nuclear receptor (NR) superfamily, and hierarchal clustering software was used to generate expression heatmaps where a red color represented upregulation (labeled Up Reg.) and a green color represented downregulation (labeled Down Reg.) when compared with hour 0 or day 0, respectively. An asterisk within the heatmap represents statistical significance (p≦0.05, Student's t-test) compared with hour or day 0. FIG. 2B contains two graphs plotting the relative expression of NRs that were upregulated at 2 hours-post osteoblastic induction. FIG. 2C is a graph plotting the relative expression of NRs that showed a biphasic response to osteoblastic induction. FIG. 2D contains two graphs plotting the relative expression of NRs that were upregulated from 6 to 24 hours-post osteoblastic induction. FIG. 2E is a graph plotting the relative expression of NRs that were downregulated from 2 to 24 hours-post osteoblastic induction. Relative expression was determined using QPCR analysis. Figure legends within each graph describe the graph labels. For clarity, SEs and p-values have been omitted but the data conform to the statistical standards of ≧1.5-fold threshold (upregulated or downregulated) and containing at least one significant time point (p≦0.05).

FIG. 3A contains a photograph of a heatmap generated from by the expression patterns of nuclear receptor genes at later stages of osteoblastic differentiation (day 7, 10, and 16 following osteoblastic induction). QPCR was performed on the 49 members of the NR superfamily, and hierarchal clustering software was used to generate expression heatmaps where a red color represented upregulation (labeled Up Reg.) and a green color represented downregulation (labeled Down Reg.) when compared with hour 0 or day 0, respectively. An asterisk within the heatmap represents statistical significance (p≦0.05, Student's t-test) compared with hour or day 0. FIG. 3B contains graphs plotting the relative expression of NRs that were upregulated at 7-16 days-post osteoblastic induction. FIG. 3C contains graphs plotting the relative expression of NRs that were downregulated at 7-16 days-post osteoblastic induction. Relative expression was determined using QPCR analysis. Figure legends within each graph describe the graph labels. For clarity, SEs and p-values have been omitted but the data conform to the statistical standards of ≧1.5-fold threshold (upregulated or downregulated) and containing at least one significant time point (p≦0.05).

FIG. 4A is a graph of the relative gene expression profile of Rorβ in primary bone marrow stromal cells (mBMSCs). Osteoblastic differentiation of mBMSCs was induced at confluence and samples harvested at 0, 7, 10, and 16 days (n=4 for each time point). QPCR was performed using primers specific for Rorβ. The data are presented as the mean±SE, and an asterisk (*) represents statistical significance of p≦0.05 (Student's t-test). FIG. 4B contains photographs of cell mineralization depicted by Alizarin red stain at the indicated time points.

FIG. 5 contains graphs of NR expression in bone marrow lin-cells in young and aged mice, as determined by QPCR analysis. Those genes exhibiting statistically significant expression changes (p≦0.05, Student's t-test) are indicated. The bars represent fold-induction relative to the young mouse cohort. The data are presented as the mean±SE, and p-values are indicated.

FIG. 6 contains a graph and photographs showing decreased Rorβ gene expression coupled with increased mineralization of MC3T3-E1 mouse osteoblasts. Parallel sets of MC3T3-E1 cells were treated for 14 days with either growth media (C), standard osteoblast differentiation media (DM) or DM supplemented with 100 ng/μg bone morphogenetic protein (BMP)-2 (DM+BMP2). Bone nodule formation was determined using Alizarin red stain and Rorβ gene expression quantified using QPCR (n=4). The data are presented as the mean±SE, and an asterisk (*) represents statistical significance of p≦0.01 (Student's t-test) compared with growth media (C) alone.

FIG. 7A is a flow chart depicting how GFP or Rorβ-GFP expression vectors were stably transfected into mouse MC3T3-E1 osteoblasts. Following two weeks of G418 antibiotic selection, the cells were sorted based on GFP positivity using fluorescence-activated cell sorting (FACS) and expanded. FIG. 7B is a graph showing Rorβ expression in MC3T3-GFP and MC3T3-Rorβ-GFP cells by QPCR analysis. FIG. 7C is a graph showing Rorβ expression by QPCR analysis in MC3T3-GFP and MC3T3-Rorβ-GFP cells that were plated and treated at confluence with either growth medium or osteoblast differentiation medium for 14 days and harvested.

FIG. 8A contains photographs of MC3T3-GFP and MC3T3-Rorβ-GFP cells that were plated and treated at confluence with either growth medium (GM) or osteoblast differentiation medium (DM) for 14 days and bone nodule formation was determined using Alizarin red staining FIG. 8B contains graphs plotting osteocalcin and osterix expression in identically treated cells that were prepared. Relative expression was assayed using QPCR (n=4). A single asterisk (*) represents significance of p≦0.01 compared to GM within each cell model, and double asterisks (**) represents significance of p≦0.01 compared to MC3T3-GFP DM (Student's t-test). FIG. 8C is a graph plotting Rorβ and oxterix relative expression in MC3T3-E1 cells that were transfected with either a non-specific siRNA control (Cont) or a mouse-specific Rorβ siRNA (Rorβ) and harvested 48 hours later. A single asterisk (*) represents significance of p≦0.01 compared to the control siRNA (Student's t-test). FIG. 8D is a graph plotting luciferase levels in U2OS cells that were transiently transfected (n=6) with the indicated plasmids and harvested 72 hours later. Luciferase and protein assays were performed. The data are presented as the mean±SE. A single asterisk (*) represents significance of p≦0.01 compared to vector alone, and double asterisks (**) represents significance of p≦0.01 compared to Runx2 alone (Student's t-test).

FIG. 9A is a schematic representation of the series of deletions made in select domains of Rorβ. Mutants were made using standard PCR techniques. The numbers represent the amino acid number located at the boundary of each deletion. The abbreviations are as follows: WT=wildtype, DBD=DNA binding domain, LBD=Ligand binding domain, AD=Activation domain. The black bar in the bottom construct represents the location of the E28A/G29A mutation. FIG. 9B contains a photograph of a Western blot and a graph plotting densitometric analysis. Western blot analysis was performed using an antibody directed against the FLAG epitope on the Rorβ species. Densitometric analysis was performed by using Lamin A/C as a nuclear protein loading control. FIG. 9C and FIG. 9D are graphs plotting activation of the luciferase reporter construct using the indicated mutants and transfection conditions.

FIG. 10A is a photograph of an immunoprecipitation detecting Runx2 in Rorβ immunoprecipitates from nuclear extracts of U20S cells. FIG. 10B is a photograph of an immunoprecipitation detecting Rorβ in Runx2 immunoprecipitates from nuclear extracts of U20S cells.

FIG. 11 contains photographs showing immunohistochemistry of Rorβ, Runx2, and merged Rorβ-Runx2 in low-density and high-density MC3T3-E1 cultures. Nuclear staining (DAPI) is also shown.

FIG. 12 is a graph plotting the expression level of the indicated genes in primary human muscle cells isolated from young (17 year old) and old (68 year old) humans. The cells were assessed as blasts or were differentiated into myotubes.

FIG. 13 is a bar graph plotting data from a human study where bone needle biopsies (1-2 mm diameter) were isolated from the posterior iliac crest in 20 young (30±5 years of age) and 20 old (73±7 years of age) women. qPCR was performed for Rorβ and a selected subset of Rorβ target genes. Similar to the data from mouse osteoblastic precursor cells (FIG. 5), Rorβ itself (1.6×) as well as multiple Rorβ target genes (P=0.001 for the pathway) were up-regulated in the biopsies from the old women.

### DETAILED DESCRIPTION

This document provides methods and materials for reducing bone loss in a mammal. For example, this document provides methods and material for using one or more inhibitors of an Rorβ polypeptide to reduce bone loss in a mammal (e.g., a human) or to increase the efficacy of a compound designed to reduce bone loss in a mammal (e.g., a human).

As described herein, one or more (e.g., one, two, three, four, or more) inhibitors of a Rorβ polypeptide can be administered to a mammal (e.g., a human) having a bone loss condition (e.g., osteoporosis) under conditions wherein the rate of bone loss is reduced or bone mass within the mammal is increased. A Rorβ polypeptide can be a human Rorβ polypeptide having the amino acid sequence set forth in GenBank® Accession No. NM_{—}006914 (GI No. 62865658). Examples of inhibitors of an Rorβ polypeptide include, without limitation, inhibitory anti-Rorβ polypeptide antibodies, siRNA molecules designed to reduce Rorβ polypeptide expression, shRNA molecules designed to reduce Rorβ polypeptide expression, nucleic acid vectors designed to express siRNA or shRNA molecules designed to reduce Rorβ polypeptide expression, and anti-sense molecules designed to reduce Rorβ polypeptide expression. In some cases, an inhibitor of a Rorβ polypeptide can be an inhibitor of Rorβ polypeptide activity. Examples of inhibitors of Rorβ polypeptide activity include, without limitation, inhibitory anti-Rorβ polypeptide antibodies. In some cases, an inhibitor of a Rorβ polypeptide can be an inhibitor of Rorβ polypeptide expression. Examples of inhibitors of Rorβ polypeptide expression include, without limitation, siRNA molecules designed to reduce Rorβ polypeptide expression, shRNA molecules designed to reduce Rorβ polypeptide expression, nucleic acid vectors designed to express siRNA or shRNA molecules designed to reduce Rorβ polypeptide expression, and anti-sense molecules designed to reduce Rorβ polypeptide expression.

In some cases, one or more (e.g., one, two, three, four, or more) inhibitors of a Rorβ polypeptide can be used as described herein to treat osteoporosis. For example, a human having osteopoprosis can be administered one or more inhibitors of a Rorβ polypeptide under conditions that result in a reduced rate of bone loss or an increase in bone mineralization. In some cases, one or more (e.g., one, two, three, four, or more) inhibitors of a Rorβ polypeptide can be used as described herein to increase the efficacy of an osteoporosis treatment. Examples of such osteoporosis treatments include, without limitation, teriparatide.

One or more of the inhibitors of a Rorβ polypeptide provided herein can be formulated into a pharmaceutical composition that can be administered to a mammal (e.g., rat, mouse, rabbit, pig, cow, monkey, or human). For example, inhibitory anti-Rorβ polypeptide antibodies can be in a pharmaceutically acceptable carrier or diluent. A “pharmaceutically acceptable carrier” refers to any pharmaceutically acceptable solvent, suspending agent, or other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, without limitation, water, saline solutions, dimethyl sulfoxide, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

The invention will be further described in the following examples, which do not limit the scope of the invention described in the claims.

### EXAMPLES

### Example 1

### Rorβ Expression is Inversely Correlated with Osteoblast Differentiation

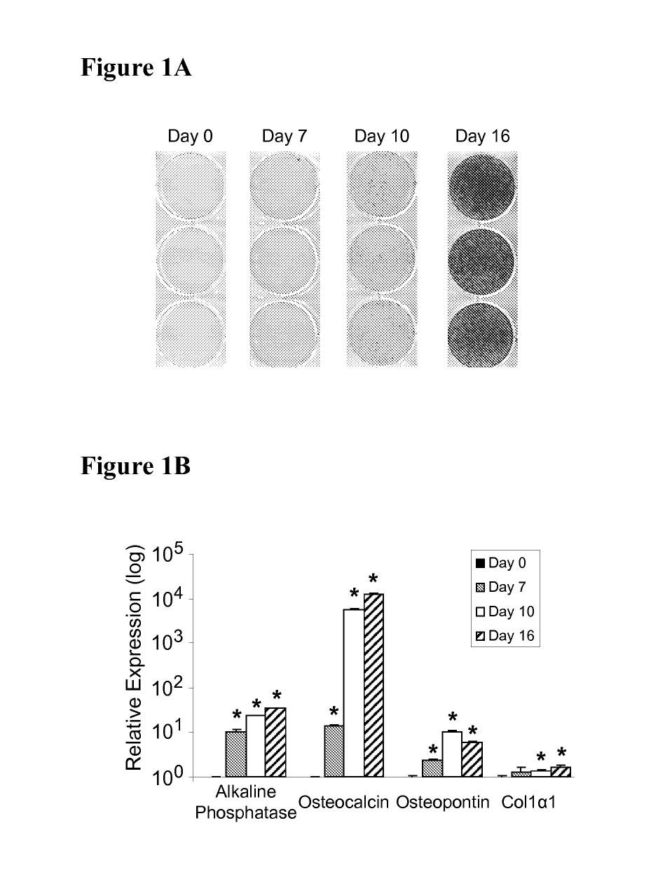

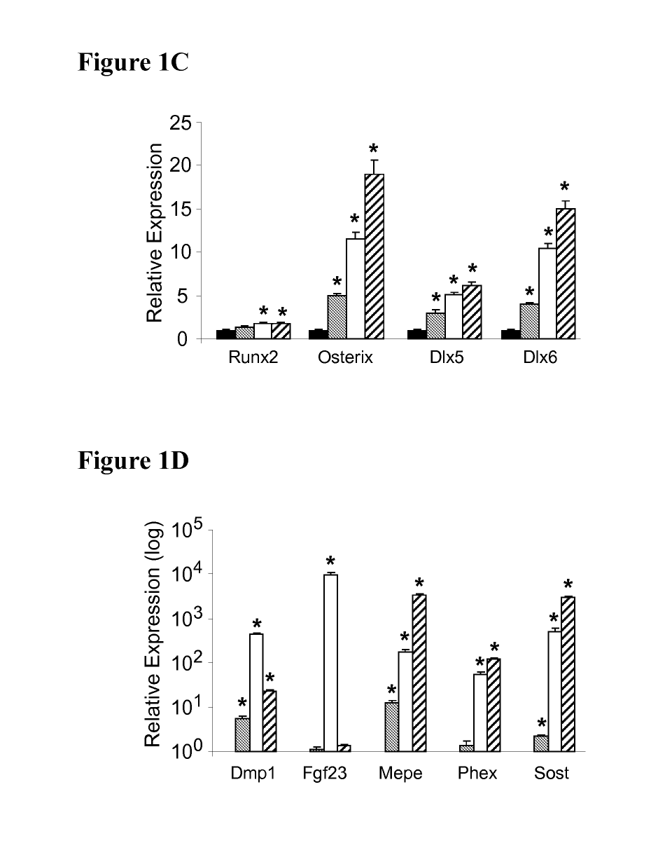

Primary mouse calvarial cells were chosen for this study since they represent a well known osteoblast model system that exhibits robust mineralization and increases in bone marker gene expression following induction of differentiation by ascorbate and β-glycerophosphate. Isolation of primary mouse calvarial osteoblasts and bone marrow stromal cells from C57BL/6 mice was performed as described elsewhere (Monroe et al., BMC Musculoskelet. Disord., 11:104-113 (2010)). Cells were maintained in αMEM growth medium (Invitrogen, Carlsbad, Calif.) supplemented with 1× antibiotic/antimycotic (Invitrogen) and 10% (v/v) fetal bovine serum (Hyclone, Logan, Utah). For the osteoblast differentiation assays, passage 4 cells were plated at a density of 10⁴ cells/cm² in 6-well plates (Corning Incorporated Life Sciences, Lowell, Mass.) in αMEM growth medium (n=4). At confluence, the media was replaced with growth medium supplemented with 50 mg/L ascorbic acid and 10 mM β-glycerophosphate (Sigma-Aldrich, St. Louis, Mo.). Differentiation was induced at confluence and samples collected at day 0, 7, 10, and 16 and assayed for calcium deposition by Alizarin red staining and osteoblast gene expression by quantitative PCR (QPCR). Alizarin red staining was done as described elsewhere (Monroe et al., BMC Musculoskelet. Disord., 11:104-113 (2010)). Briefly, cells were washed in 1×PBS and fixed in 3.75% paraformaldehyde overnight at room temperature. Following two 1×PBS washes, the cells were stained with 1.2% Alizarin red (v/v) (Sigma-Aldrich) pH 4.2 for 20 minutes. The cells were extensively washed with 1×PBS and scanned. Robust Alizarin red-positive nodule formation was observed at day 10 and 16 following the induction of differentiation, indicative of a highly mineralizing cell population (FIG. 1A). Gene expression profiles of classic osteoblast marker genes (alkaline phosphatase, osteocalcin, osteopontin and collagen, type I, alpha (Col1α1)) involved in formation of the secreted glycoprotein matrix were increased compared to day 0 (FIG. 1B), confirming the production of a highly osteogenic cell population. Transcriptional regulation of osteoblastic differentiation is a tightly controlled process involving Runx2, osterix, and Dlx5/6, and expression of these genes was increased at nearly all time points (FIG. 1C). Due to the high expression of these bone marker genes at day 16, it was surmised that these cells may be starting to exhibit an osteocytic phenotype. Remarkably, large increases in the well-established osteocytic markers dentin matrix protein 1 (Dmp1), fibroblast growth factor 23 (Fgf23), matrix extracellular phosphoglycoprotein (Mepe), phosphate regulating endopeptidase homolog, X-linked (Phex), and sclerostin (Sost) were observed at the later stages of differentiation (FIG. 1D). Collectively, these data indicate that differentiation of primary mouse calvarial cells led to marked increases in mineralization, as well as activation of genes involved in osteoblast and osteocyte biology.

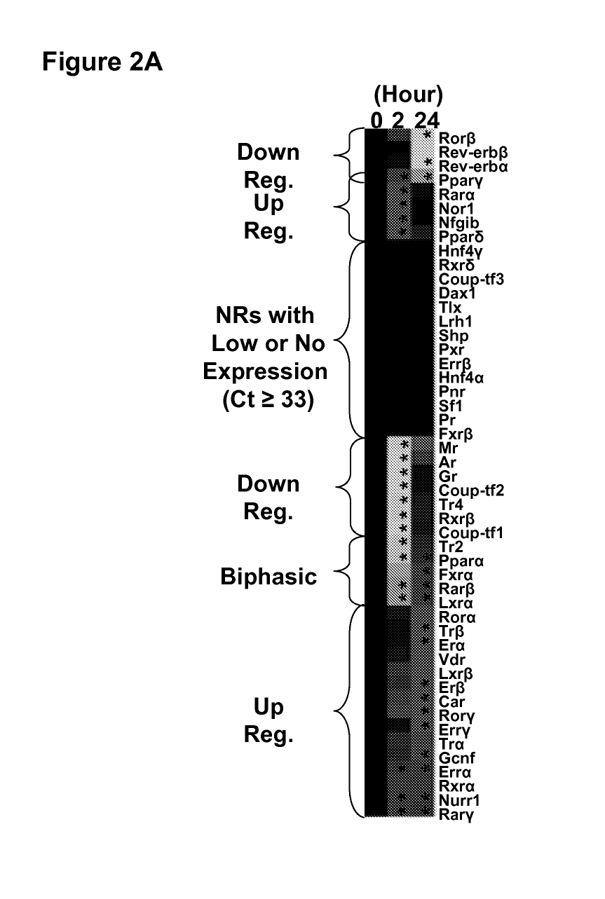

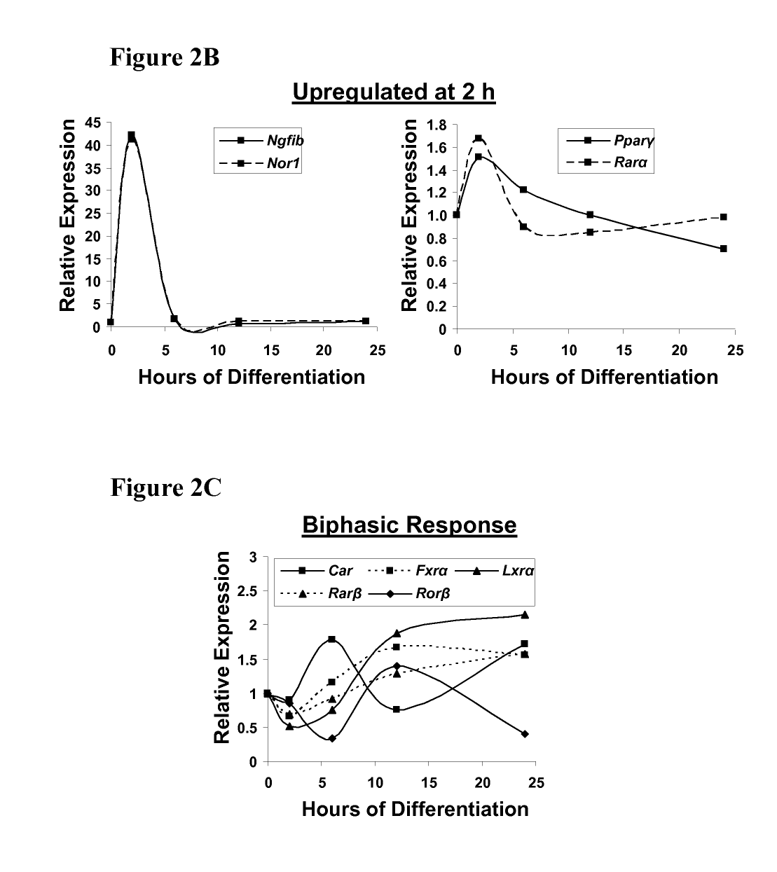

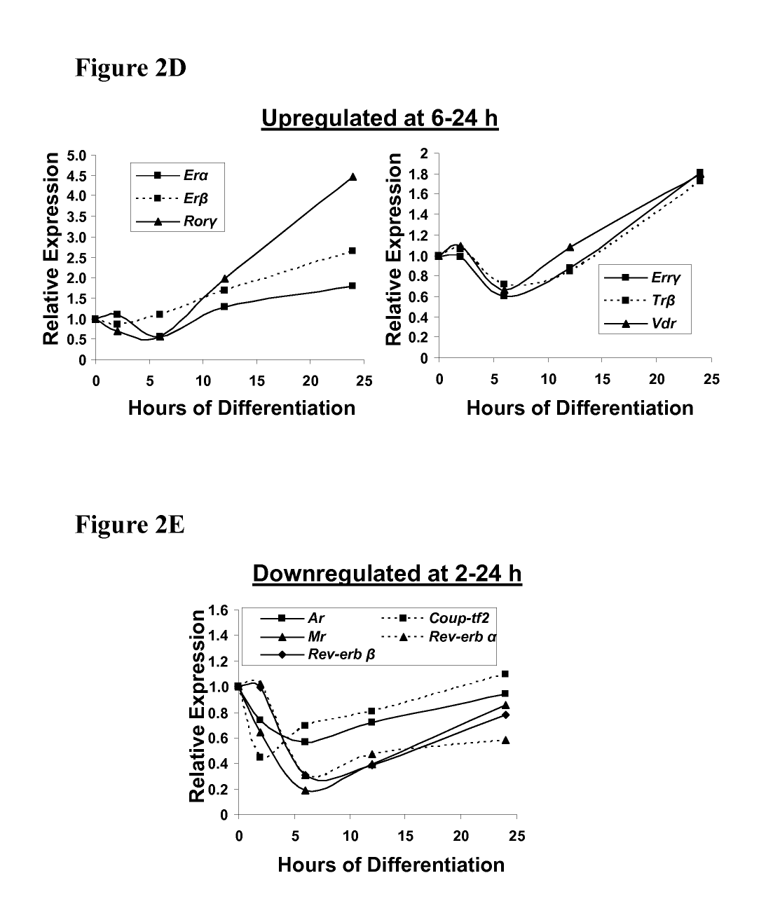

As a first step to understanding the influence of osteoblastic differentiation on NR gene expression, the mRNA expression patterns of the 49 NRs were examined during the first 24 hours following addition of osteoblast differentiation media. Analysis of the entire NR superfamily revealed that 35 were expressed at either 2 or 24 hours following osteoblastic induction; whereas 14 were not expressed at any time point using a threshold cutoff of Ct≧33 (Fu et al., Mol. Endocrinol., 19:2437-2450 (2005)). The expression patterns of the NRs were subjected to hierarchal cluster analysis, and a heatmap was generated, further subdividing the genes as either upregulated or downregulated (FIG. 2A). Unsupervised cluster analysis was performed essentially as described elsewhere (Xie et al., Mol. Endocrinol., 23:724-733 (2009)). Briefly, the mean gene expression fold-changes for each gene were adjusted using log transformation to center the data around 0 and normalized to set the magnitude of the control value (day 0) for each gene to 1 using Gene Cluster 3.0 (http at “://ranalbl.gov/eisen/”). The adjusted data were clustered by calculating Pearson's centered correlation coefficients followed by average linkage analysis in the Gene Cluster 3.0 program. Expression heatmaps, which visually describe the cluster results, were generated using TreeView (http at “://ranalbl.gov/eisen/”). The shades were labeled to indicate those genes that were upregulated and those genes that were downregulated relative to the control value (FIG. 2A). Those genes exhibiting statistical significance (p≦0.05) and a fold-change ≧1.5 compared to time 0 were reassayed using QPCR on an expanded time course (0, 2, 6, 12, and 24 hours) to generate a more detailed profile of gene expression. Comparison of the temporal patterns of NR gene expression revealed a group of four transiently upregulated genes at 2 hours, including nerve growth factor-induced gene B (Ngfib), neuron-derived orphan receptor 1 (Nor1), peroxisome proliferator-activated receptor gamma (Pparγ), and retinoic acid receptor alpha (Rarα) (FIG. 2B). It is of interest that Pparγ, the main controller of adipogenesis, was induced at 2 hours followed by downregulation by 24 hours of osteogenic media treatment. Another group of genes exhibited biphasic expression during the time course, which includes liver X receptor alpha (Lxrα), Rarβ, farnesoid X receptor (Fxrα), constitutive androstane receptor (Car), and retinoic acid receptor-related orphan receptor beta (Rorβ) that were suppressed at 2 hours and upregulated at 24 hours (FIG. 2B). The following genes were upregulated primarily at 24 hours: vitamin D receptor (Vdr), Rorγ, estrogen-related receptor gamma (Errγ), thyroid hormone receptor beta (Trβ), and estrogen receptor (Er)-α and -β (FIG. 2D). The genes transiently downregulated at 2 hours included Rev-erb-α/β, chicken ovalbumin upstream promoter transcription factor 2 (Couptf2), and classic group 3 receptors such as androgen receptor (Ar) and mineralocorticoid receptor (Mr) (FIG. 2E). Collectively, these data demonstrate that significant NR gene expression changes occur very early in the process of osteoblastic differentiation that may set the stage for modulated sensitivity to specific hormonal or nutritional influences.

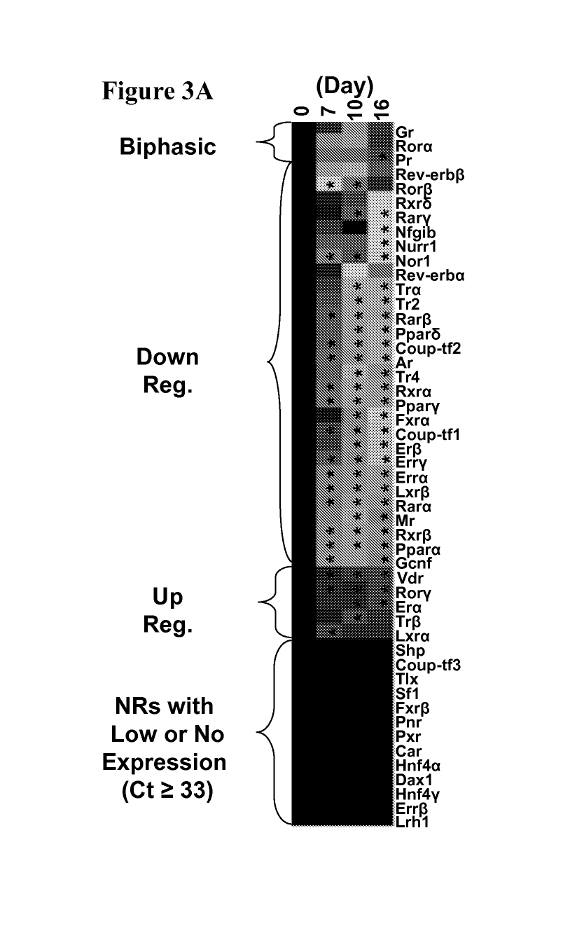

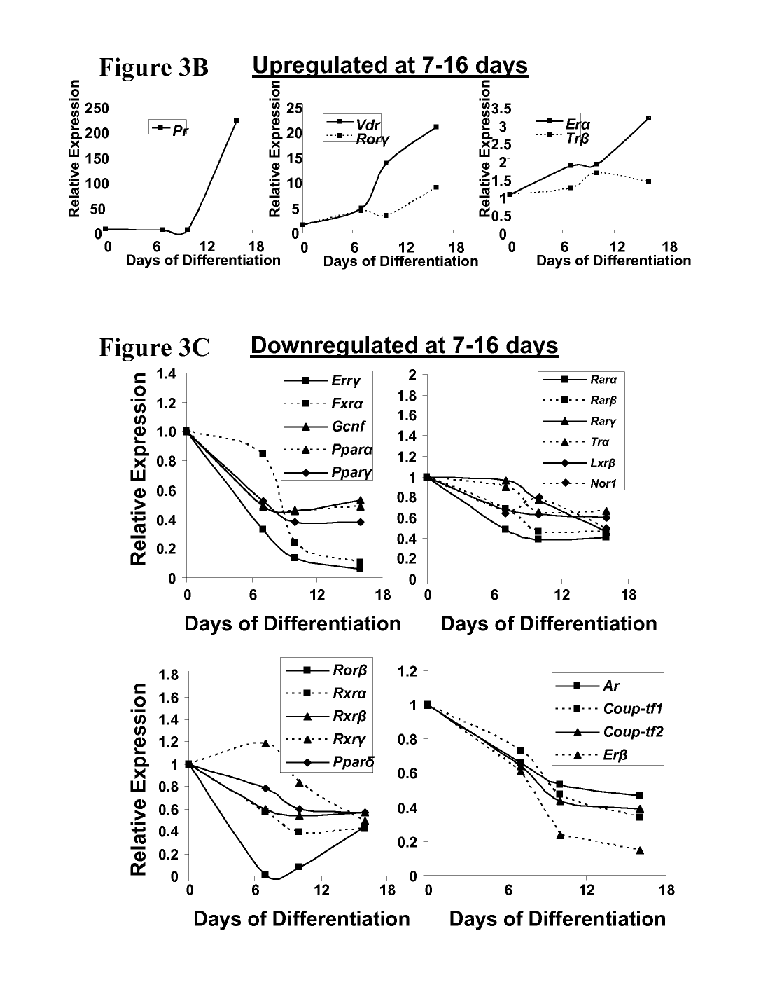

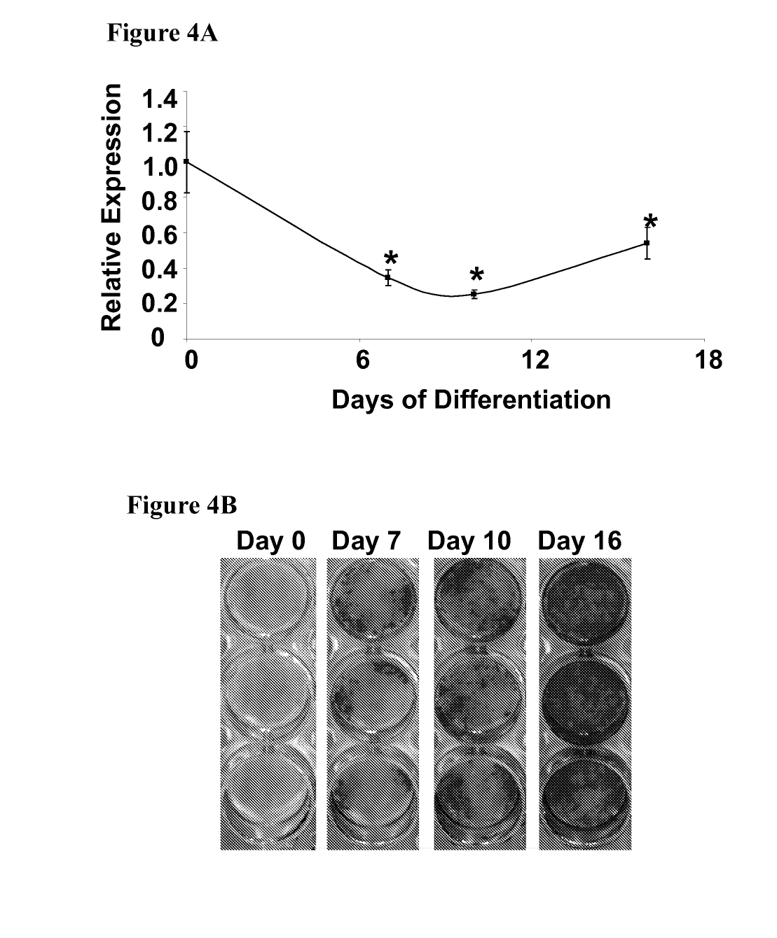

To characterize transcriptional regulation of NR expression at later stages of osteoblastic differentiation, the expression patterns of the NRs at 7, 10, and 16 days following osteoblastic induction were determined using QPCR. The data were subjected to hierarchal cluster analysis, and a heatmap was generated (FIG. 3A). Examination of the temporal expression pattern in late differentiating osteoblasts revealed that 36 were expressed late in osteoblastic differentiation, whereas 13 were not expressed. Further examination of the expressed NRs revealed that only 5 genes (Pr, Vdr, Rorγ, Erα, and Trβ) were significantly upregulated late in differentiation using the expanded time course (FIG. 3B). Pr was induced 217-fold at day 16 but was undetectable at all earlier time points, suggesting that Pr may have functions limited to the late-osteoblastic and/or osteocytic phenotype. Upregulation of Vdr (20-fold), Erα (3.1-fold), and Trβ (1.6-fold) which have reported roles in bone biology, was also observed late in osteoblastic differentiation. Rorγ, whose role in osteoblast differentiation is unknown, was upregulated 8.4-fold. Most remarkable was the observation that 78% (28/36) of the expressed genes were downregulated at the later time points including all members of the Rar (NR1B), Rxr (NR2B), and Ppar (NR1C) gene families, which have roles in the support of adipogenesis. Strikingly, significant downregulation of Errγ (16-fold), Fxrα (9.5-fold), Ar (2.1-fold), and Erβ (6.7-fold) was observed, some of which have functions in osteoblasts (FIG. 3C). Of particular interest, Rorβ expression drastically declined to undetectable levels at day 7-10 and returned to about 40% of control at day 16. A similar temporal Rorβ expression profile was observed in primary bone marrow stromal cells (FIG. 4). Rorβ gene expression was not detectable in osteoclast cultures (data not shown). Collectively, these data clearly demonstrate that significant changes in NR expression occur during late osteoblastic differentiation, mostly downregulation, which may be important in osteoblast and/or osteocyte function.

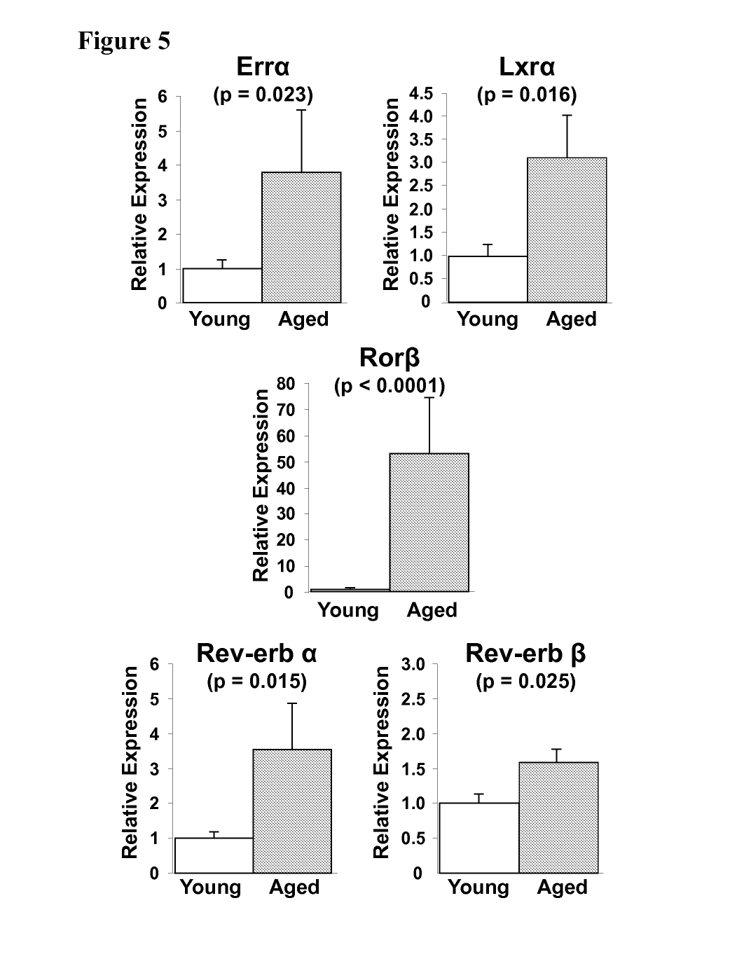

Although characterization of NR gene expression during calvarial osteoblast differentiation yielded interesting and important results regarding the function of these receptors in this in vitro system, understanding which NRs are important in regulating osteogenesis in a more physiological system was imperative. NR gene expression changes associated with age-related bone loss was therefore characterized in mice. A study by Syed et al. (J. Bone Miner. Res., 25:2438-2446 (2010)) analyzed the bone marker gene expression patterns of cells isolated from the lineage negative (lin-) population in femoral bone marrow of young (6 month) and aged (18-22 month) mice. In that study, the aged mice had marked reductions in bone mass and in osteoblast numbers on bone-surfaces, consistent with an age-related impairment in osteogenesis. The bone marrow lin-cells represent a population depleted of the hematopoietic cell lineage, which have been shown to be highly enriched for osteoprogenitor cells which mineralize in vitro, form bone in vivo, and express bone-related genes, thereby providing a useful cell population for evaluation of effects of aging on osteoblast progenitor cells. Therefore, the QPCR methodology described below was applied to these samples (n=7-8). Surprisingly, only 5 NRs were found to exhibit statistically significant gene expression changes in aged mice when compared to young mice (FIG. 5).

Total cellular RNA was harvested at the indicated times following induction of osteoblast differentiation using QIAzol Lysis Reagent and RNeasy Mini Columns (Qiagen, Valencia, Calif.). DNase treatment was performed to degrade potential contaminating genomic DNA using an on-column RNase-free DNase solution (Qiagen). Three μg of total RNA was used in a reverse transcriptase (RT) reaction using the High Capacity cDNA Reverse Transcription Kit (Applied Biosystems by Life Technologies, Foster City, Calif.) according to manufacturer instructions. The RT reactions were diluted 1:5 and 1 μL used in a 10 μL total reaction volume for real-time quantitative PCR (QPCR) using the QuantiTect SYBR Green PCR Kit (Qiagen) and the ABI 7900HT Fast Real-Time PCR System (Applied Biosystems). All primers were designed using Primer Express® Software Version 3.0 (Applied Biosystems). The nuclear receptor, bone marker, and reference gene primer sequences used are set forth in Tables 1-3, respectively.

TABLE 1
Primer sequences of the 49 members of the nuclear
hormone receptor superfamily in mouse.

| Common | Formal | | | Amplicon |
|---|---|---|---|---|
| Name | Name | Accession# | QPCR Primers (5′-3′) | Length |
| DAX-1 | NR0B1 | NM_007430 | GCCCAAGATCACCTGCACTT (SEQ ID NO: 1) | 62 |
| | | | ATTTCCTGCGTCGTGTTGGT (SEQ ID NO: 2) | |
| SHP | NR0B2 | NM_011850 | CCTATCATGGGAGACGTTGACA (SEQ ID NO: 3) | 63 |
| | | | GGGTCACCTCAGCAAAAGCA (SEQ ID NO: 4) | |
| TRα | NR1A1 | NM_178060 | TCAACCACCGCAAACACAAC (SEQ ID NO: 5) | 60 |
| | | | CAGTCACCTTCATCAGCAGCTT (SEQ ID NO: 6) | |
| TRβ | NR1A2 | NM_001113417 | AGCCAAGCGGAAGCTTATAGAG (SEQ ID NO: 7) | 78 |
| | | | GGCTTGTGCCCAATTGATTT (SEQ ID NO: 8) | |
| RARα | NR1B1 | NM_009024 | AAGGTGGACATGCTGCAAGAG (SEQ ID NO: 9) | 62 |
| | | | CTCCGTTTCCGGACGTAGAC (SEQ ID NO: 10) | |
| RARβ | NR1B2 | NM_011243 | CTGACCTTGTGTTCACCTTTGC (SEQ ID NO: 11) | 66 |
| | | | GGCCTGTTTCTGTGTCATCCA (SEQ ID NO: 12) | |
| RARγ | NR1B3 | NM_011244 | GACCAGATCACGCTGCTCAA (SEQ ID NO: 13) | 63 |
| | | | CCTTGTACAGATCCGCAGCAT (SEQ ID NO: 14) | |
| PPARα | NR1C1 | NM_011144 | GGATTGTGCACGTGCTTAAGC (SEQ ID NO: 15) | 65 |
| | | | TGGGAAGAGGAAGGTGTCATCT (SEQ ID NO: 16) | |
| PPARδ | NR1C2 | NM_011145 | GAAGCCATCCAGGACACCAT (SEQ ID NO: 17) | 60 |
| | | | AGGGTGGTTGACCTGCAGAT (SEQ ID NO: 18) | |
| PPARγ | NR1C3 | NM_011146 | CCCACCAACTTCGGAATCAG (SEQ ID NO: 19) | 58 |
| | | | AATGCGAGTGGTCTTCCATCA (SEQ ID NO: 20) | |
| REV- | NR1D1 | NM_145434 | GCTCCATCGTTCGCATCAAT (SEQ ID NO: 21) | 69 |
| ERBα | | | TGCCAACGGAGAGACACTTCT (SEQ ID NO: 22) | |
| REV- | NR1D2 | NM_011584 | GCAATCCCAAGAACGCTGAT (SEQ ID NO: 23) | 64 |
| ERBβ | | | CAATCTGTGCGGTCACTCTTCA (SEQ ID NO: 24) | |
| RORα | NR1F1 | NM_013646 | CTCGAGATGCTGTCAAGTTTGG (SEQ ID NO: 25) | 60 |
| | | | CGGCGTACAAGCTGTCTCTCT (SEQ ID NO: 26) | |
| RORβ | NR1F2 | NM_001043354 | CGGGATCCACTACGGAGTCA (SEQ ID NO: 27) | 62 |
| | | | GCTGGCTCCTCCTGAAGAATC (SEQ ID NO: 28) | |
| RORγ | NR1F3 | NM_011281 | CTCAGCGCCCTGTGTTTTTC (SEQ ID NO: 29) | 58 |
| | | | TGAGAACCAGGGCCGTGTAG (SEQ ID N0: 30) | |
| LXRβ | NR1H2 | NM_009473 | AAGGCGTCCACCATTGAGAT (SEQ ID NO: 31) | 68 |
| | | | ATGCATTCTGTCTCGTGGTTGT (SEQ ID NO: 32) | |
| LXRα | NR1H3 | NM_013839 | CACGCCTACGTCTCCATCAA (SEQ ID NO: 33) | 57 |
| | | | TAGCATCCGTGGGAACATCA (SEQ ID NO: 34) | |
| FXRα | NR1H4 | NM_001163700 | TGCTCACAGCGATCGTCATC (SEQ ID NO: 35) | 62 |
| | | | CACCGCCTCTCTGTCCTTGA (SEQ ID NO: 36) | |
| FXRβ | NR1H5 | NM_198658 | GGGACTCCCAGGATTTGAAAA (SEQ ID NO: 37) | 64 |
| | | | TTTTGACGCCTTCTGTAATGCA (SEQ ID NO: 38) | |
| VDR | NR1I1 | NM_009504 | GGCTTCCACTTCAACGCTATG (SEQ ID NO: 39) | 51 |
| | | | CATGCTCCGCCTGAAGAAAC (SEQ ID NO: 40) | |
| PXR | NR1I2 | NM_010936 | GGAAGAGCCCATCAACGTAGAG (SEQ ID NO: 41) | 62 |
| | | | CCCCACATACACGGCAGATT (SEQ ID NO: 42) | |
| CAR | NR1I3 | NM_009803 | AGACGAACAGTCAGCAAAACCA (SEQ ID NO: 43) | 60 |
| | | | GACCTCACACCTTCCAGCAAA (SEQ ID NO: 44) | |
| HNF4α | NR2A1 | NM_008261 | GCCGACAATGTGTGGTAGACA (SEQ ID NO: 45) | 74 |
| | | | AGCCCGGAAGCACTTCTTAAG (SEQ ID NO: 46) | |
| HNF4γ | NR2A2 | NM_013920 | AAAAGAAGCGGTGCAAAATGA (SEQ ID NO: 47) | 67 |
| | | | GTTGCTGCCCTCGTAGGTACTT (SEQ ID NO: 48) | |
| RXRα | NR2B1 | NM_011305 | GCCATCTTTGACAGGGTGCTA (SEQ ID NO: 49) | 69 |
| | | | CTCCGTCTTGTCCATCTGCAT (SEQ ID N0: 50) | |
| RXRβ | NR2B2 | NM_011306 | CGCCTCACTGGAGACCTATTG (SEQ ID NO: 51) | 71 |
| | | | GTAACAGCAGCTTGGCAAACC (SEQ ID NO: 52) | |
| RXRγ | NR2B3 | NM_009107 | GCCCGTGGAGAGGATTCTAGA (SEQ ID NO: 53) | 71 |
| | | | CGTTCATGTCACCGTAGGATTC (SEQ ID NO: 54) | |
| TR2 | NR2C1 | NM_011629 | AGCGAGTCGCACGTAGCTTT (SEQ ID NO: 55) | 59 |
| | | | TTCAGGTACTCGGGCATAGGA (SEQ ID NO: 56) | |
| TR4 | NR2C2 | NM_011630 | AGTGACCTCTTTGGCCAACCT (SEQ ID NO: 57) | 65 |
| | | | GGCTGCATTTCTGAAGCATCA (SEQ ID NO: 58) | |
| TLX | NR2E1 | NM_15229 | CCCAAGTATCCCCATGAAGTGA (SEQ ID NO: 59) | 91 |
| | | | AGAGAAGCCTGGCAGCTGATT (SEQ ID NO: 60) | |
| PNR | NR2E3 | NM_013708 | CATGGGCCACCACTTTATGG (SEQ ID NO: 61) | 67 |
| | | | GTCCTCTGGCTCCAGTTTAGCA (SEQ ID NO: 62) | |
| COUP- | NR2F1 | NM_010151 | CGGTTCAGCGAGGAAGAATG (SEQ ID NO: 63) | 66 |
| TF1 | | | CCCCGTTTGTGAGTGCATACT (SEQ ID NO: 64) | |
| COUP- | NR2F2 | NM_009697 | GTTTTTCGTCCGTTTGGTAGGT (SEQ ID NO: 65) | 71 |
| TF2 | | | TGCTGCCGGACAGTAACATATC (SEQ ID NO: 66) | |
| COUP- | NR2F6 | NM_010150 | AGGTGGATGCTGCGGAGTAC (SEQ ID NO: 67) | 71 |
| TF3 | | | AGAAAGGCCACAGGCATCAG (SEQ ID NO: 68) | |
| ERα | NR3A1 | NM_007956 | ATGATGAAAGGCGGCATACG (SEQ ID NO: 69) | 64 |
| | | | TCTGACGCTTGTGCTTCAACAT (SEQ ID NO: 70) | |
| ERβ | NR3A2 | NM_207707 | CATCAGTAACAAGGGCATGGAA (SEQ ID NO: 71) | 67 |
| | | | GTCGTACACCGGGACCACAT (SEQ ID NO: 72) | |
| ERRα | NR3B1 | NM_007953 | TACGGTGTGGCATCCTGTGA (SEQ ID NO: 73) | 59 |
| | | | CTCCCCTGGATGGTCCTCTT (SEQ ID NO: 74) | |
| ERRβ | NR3B2 | NM_011934 | TTTCCCCACCTGCTAAAAAGC (SEQ ID NO: 75) | 68 |
| | | | CTTGTCCTGCTCAACCCCTAGT (SEQ ID NO: 76) | |
| ERRγ | NR3B3 | NM_011935 | GATCCCCAGACCAAGTGTGAA (SEQ ID NO: 77) | 68 |
| | | | TCGCCACACACTAAGCACAGT (SEQ ID NO: 78) | |
| GR | NR3C1 | NM_008173 | CGGTGGCAGTGTGAAATTGTA (SEQ ID NO: 79) | 64 |
| | | | CTCCAAATCCTGCAAGATGTCA (SEQ ID NO: 80) | |
| MR | NR3C2 | NM_001083906 | GCTCCCCCAGTGTTGAAAATAG (SEQ ID NO: 81) | 79 |
| | | | CTTGAAAGAGGAGAGCCCACAT (SEQ ID NO: 82) | |
| PR | NR3C3 | NM_008829 | CTGTCACTATGGCGTGCTTACC (SEQ ID NO: 83) | 112 |
| | | | TTATGCTGCCCTTCCATTGC (SEQ ID NO: 84) | |
| AR | NR3C4 | NM_013476 | TGACAACAACCAACCAGATTCC (SEQ ID NO: 85) | 65 |
| | | | GCCTCTCTCCAAGCTCATTGA (SEQ ID NO: 86) | |
| NGFIB | NR4A1 | NM_010444 | GTGTTGATGTTCCCGCCTTT (SEQ ID NO: 87) | 62 |
| | | | CCCGTGTCGATCAGTGATGA (SEQ ID NO: 88) | |
| NURR1 | NR4A2 | NM_013613 | GCGCTTAGCATACAGGTCCAA (SEQ ID NO: 89) | 61 |
| | | | GACCACCCCATTGCAAAAGAT (SEQ ID NO: 90) | |
| NOR1 | NR4A3 | NM_015743 | CAGTGTCGGGATGGTTAAGGAA (SEQ ID NO: 91) | 71 |
| | | | CAGACGACCTCTCCTCCCTTT (SEQ ID NO: 92) | |
| SF1 | NR5A1 | NM_139051 | CTGTGCGTGCTGATCGAATG (SEQ ID NO: 93) | 67 |
| | | | GCCCGGTCTCTCTTGTACATG (SEQ ID NO: 94) | |
| LRH-1 | NR5A2 | NM_010264 | TCTGCACCAGGGTCAGAGACT(SEQ ID NO: 95) | 63 |
| | | | ACGTTTTTCCCGGAGTTGTTC (SEQ ID NO: 96) | |
| GCNF | NR6A1 | NM_010264 | TCAAGAGGAGCATTTGCAACA (SEQ ID NO: 97) | 69 |
| | | | TCCGGGACATGACACAGTTCT (SEQ ID NO: 98) | |

TABLE 2
Primer sequences and functional categorization of osteoblast-
or osteocyte- marker genes.

| | | | | Amplicon |
|---|---|---|---|---|
| Name | Function | Accession # | QPCR Primers (5′-3′) | Length |
| Alkaline | Enzyme | NM_007431 | CACAGATTCCCAAAGCACCT (SEQ ID NO: 99) | 99 |
| Phosphatase | | | GGGATGGAGGAGAGAAGGTC (SEQ ID NO: 100) | |
| Hormone, | Bone Marker | NM_007541 | CCTGAGTCTGACAAAGCCTTCA (SEQ ID NO: 101) | 63 |
| Osteocalcin | | | GCCGGAGTCTGTTCACTACCTT (SEQ ID NO: 102) | |
| Extracellular | Matrix Protein | NM_007742 | GCTTCACCTACAGCACCCTTGT (SEQ ID NO: 103) | 66 |
| Collagen1α1 | | | TGACTGTCTTGCCCCAAGTTC (SEQ ID NO: 104) | |
| Extracellular | Matrix Protein | NM_009263 | CCCGGTGAAAGTGACTGATTCT (SEQ ID NO: 105) | 62 |
| Osteopontin | | | GATCTGGGTGCAGGCTGTAAA (SEQ ID NO: 106) | |
| Extracellular | Matrix Protein | NM_009242 | GAGGAGGTGGTGGCTGACAA (SEQ ID NO: 107) | 37 |
| Osteonectin | | | CACCTTGCCATGTTTGCAAT (SEQ ID NO: 108) | |
| Runx2 | Transcriptional | NM_009820 | GGCACAGACAGAAGCTTGATGA (SEQ ID NO: 109) | 72 |
| | Regulation | | GAATGCGCCCTAAATCACTGA (SEQ ID NO: 110) | |
| Osterix | Transcriptional | NM_130458 | GGAGGTTTCACTCCATTCCA (SEQ ID NO: 111) | 103 |
| | Regulation | | TAGAAGGAGCAGGGGACAGA (SEQ ID NO: 112) | |
| DIx5 | Transcriptional | NM_198854 | TCTCAGGAATCGCCAACTTTG (SEQ ID NO: 113) | 66 |
| | Regulation | | CGCGGGACTGTAGTAGTCAGAA (SEQ ID NO: 114) | |
| DIx6 | Transcriptional | NM_010057 | GGGACGACACAGATCAACAAAA (SEQ ID NO: 115) | 67 |
| | Regulation | | CCCTTTCCGTTGAACCTGATT (SEQ ID NO: 116) | |
| Dmp1 | Osteocyte | NM_016779 | TGCTCTCCCAGTTGCCAGAT (SEQ ID NO: 117) | 77 |
| | Marker | | AATCACCCGTCCTCTCTTCAGA (SEQ ID NO: 118) | |
| Fgf23 | Osteocyte | NM_022657 | TCTCCACGGCAACATTTTTG (SEQ ID NO: 119) | 57 |
| | Marker | | CTGGCGGAACTTGCAATTCT(SEQ ID NO: 120) | |
| Mepe | Osteocyte | NM_053172 | TGCTGCCCTCCTCAGAAATATC (SEQ ID NO: 121) | 47 |
| | Marker | | GTTCGGCCCCAGTCACTAGA (SEQ ID NO: 122) | |
| Phex | Osteocyte | NM_011077 | CCTTGGCTGAGACACAATGTTG (SEQ ID NO: 123) | 67 |
| | Marker | | GCCTTCGGCTGACTGATTTCT (SEQ ID NO: 124) | |
| Sost | Osteocyte | NM_024449 | ACTTGTGCACGCTGCCTTCT (SEQ ID NO: 125) | 74 |
| | Marker | | TGACCTCTGTGGCATCATTCC (SEQ ID NO: 126) | |

TABLE 3
Primer sequences of QPCR reference genes.

| | | | Amplicon |
|---|---|---|---|
| Name | Accession # | QPCR Primers (5′-3′) | Length |
| 18S RNA | NR_003278 | AGTCCCTGCCCTTTGTACACA (SEQ ID NO: 127) | 56 |
| | | GGCCTCACTAAACCATCCAATC (SEQ ID NO: 128) | |
| B2m | NM_009735 | CACTGACCGGCCTGTATGCTA (SEQ ID NO: 129) | 61 |
| | | TGGGTGGCGTGAGTATACTTGA (SEQ ID NO: 130) | |
| RpL13A | NM_009438 | GTGGTCCCTGCTGCTCTCAA (SEQ ID NO: 131) | 67 |
| | | CCCCAGGTAAGCAAACTTTCTG (SEQ ID NO: 132) | |
| Tbp | NM_013684 | GCCTTACGGCACAGGACTTACT (SEQ ID NO: 133) | 152 |
| | | GCTGTCTTTGTTGCTCTTCCAA (SEQ ID NO: 134) | |
| Gapdh | NM_008084 | GGGAAGCCCATCACCATCTT (SEQ ID NO: 135) | 47 |
| | | GCCTCACCCCATTTGATGTT (SEQ ID NO: 136) | |
| G6pdx | NM_008062 | GGAGGAGTTCTTTGCCCGTAA (SEQ ID NO: 137) | 67 |
| | | GTGCTTATAGGAGGCTGCATCA (SEQ ID NO: 138) | |
| Polr2a | NM_009089 | CGAATTGACTTGCGTTTCCA (SEQ ID NO: 139) | 74 |
| | | ATGTGCCGTTCCACCTTATAGC (SEQ ID NO: 140) | |
| Tuba1a | NM_011653 | GGTTCCCAAAGATGTCAATGCT (SEQ ID NO: 141) | 62 |
| | | CAAACTGGATGGTACGCTTGGT (SEQ ID NO: 142) | |
| Hprt | NM_013556 | CGTGATTAGCGATGATGAACCA (SEQ ID NO: 143) | 75 |
| | | TCCAAATCCTCGGCATAATGA (SEQ ID NO: 144) | |

Normalization for variations in input RNA was performed using a panel of 9 reference genes (18S, glucose-6-phosphate dehydrogenase (G6pdh), glyceraldehyde-3-phosphate dehydrogenase (Gapdh), hypoxanthine guanine phosphoribosyl transferase (Hprt), ribosomal protein L13A (Rpl13A), polymerase (RNA) II (DNA directed) polypeptide A (Polr2a), TATA binding protein (Tbp), tubulin alpha 1a (Tubα1a), and β2-microglobulin (B2m)) using the geNorm algorithm to select the three most stable reference genes. The PCR Miner algorithm was used to correct for variations in amplification efficiencies. The median cycle threshold (Ct) for each gene in each sample was normalized to the geometric mean of the median Ct of the reference genes as determined by the geNorm algorithm using the formula: 2^{(reference Ct-gene of interest Ct)}. The resulting ΔCt for each gene was used to calculate relative gene expression changes between samples. Analysis of gene expression in hematopoietic lineage negative (lin-) bone marrow cells, a mesenchymal-enriched cell fraction from young (6 month), and aged (18-22 month) mice (FIG. 5) was performed using cDNA samples (n=7-8) derived from another study using the methods described elsewhere (Syed et al., J. Bone Miner. Res., 25:2438-2446 (2010)).

Significant upregulation of Errα (3.8-fold), Lxrα (3.1-fold), Rev-erbα (3.6-fold), and Rev-erbβ (1.6-fold) was observed (FIG. 5). Interestingly, all these NRs are associated with either age-related bone loss or the support of adipogenesis. Rorβ, a gene originally thought to have actions limited to the central nervous system, retina, and circadian rhythms, was induced 53-fold.

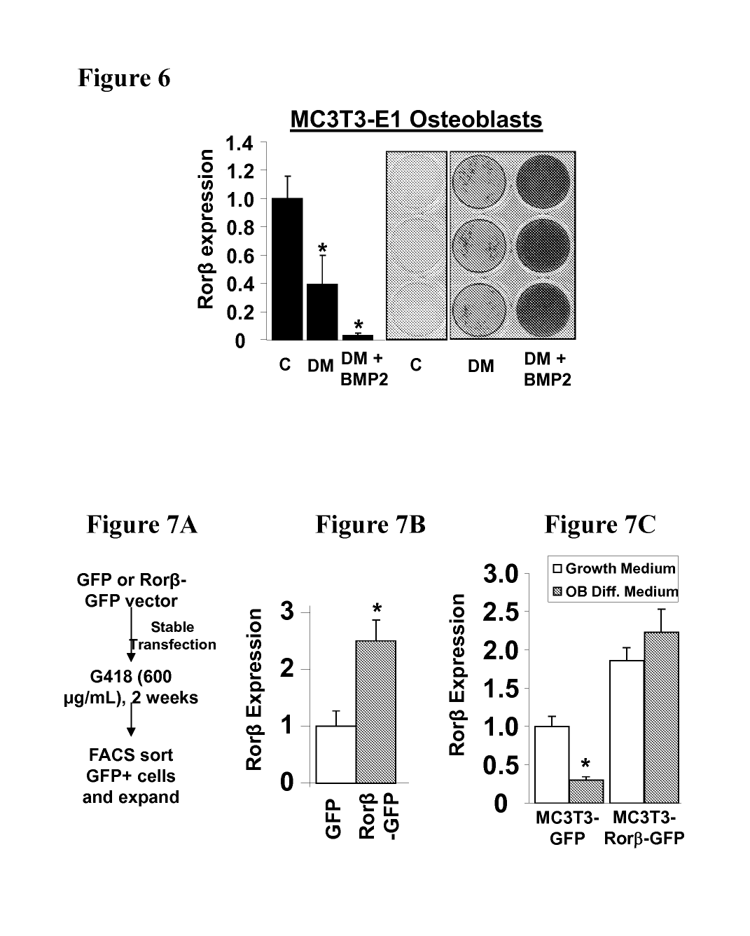

Examination of the calvarial osteoblast and aged mouse datasets revealed that Rorβ exhibits a gene expression pattern inversely correlated with osteogenic potential in both models. Therefore, to verify this phenomenon in an independent system, the mouse MC3T3-E1 osteoblastic cell line was used. MC3T3-E1 mouse osteoblasts were cultured and treated identically to the primary mouse calvarial osteoblasts except the osteoblast differentiation medium was supplemented with 100 μg/mL recombinant Bmp2 (R&D Systems, Minneapolis, Minn.) unless otherwise noted. MC3T3-E1 cells were differentiated with or without 100 ng/mL Bmp2 (to strongly induce osteoblastic differentiation in this model) for 14 days. Rorβ expression declined 2.5- and 20-fold with differentiation media (DM) or DM+Bmp2, respectively (FIG. 6). It is interesting that the amount of mineral formed and the degree of Rorβ suppression were also inversely correlated (e.g., more Rorβ suppression associated with more robust mineralization).

The dramatically decreased levels of Rorβ levels during osteoblastic differentiation of both calvarial and MC3T3-E1 osteoblasts and aged mice indicates that Rorβ expression is inversely correlated with osteogenic potential and further suggests that suppression of Rorβ may be a prerequisite for osteoblastic mineralization to occur.

### Example 2

### Rorβ Expression in MC3T3-E1 Cells Results in Impaired Mineralization and Suppressed Runx2 Function

To directly test whether Rorβ inhibits cell mineralization, two cell models were produced in MC3T3-E1 mouse osteoblasts which stably express either GFP (control) or Rorβ-GFP. A Rorβ expression construct, pCMV6-Rorβ (Origene, Rockville, Md.), was cloned as an EcoRI/MluI fragment into a vector coexpressing GFP under the control of an internal ribosome entry sequence. A vector expressing only GFP was used as a control. These vectors were electroporated into MC3T3-E1 cells using the Neon Transfection System (Invitrogen) and selected with 400 μg/mL G418 antiobiotic (Invitrogen). Following 2 weeks of cell selection and expansion, fluorescence-activated cell sorting was used to isolate the GFP-expressing population, and the cells were again expanded resulting in the MC3T3-GFP (control) and MC3T3-Rorβ-GFP cell models (FIG. 7A).

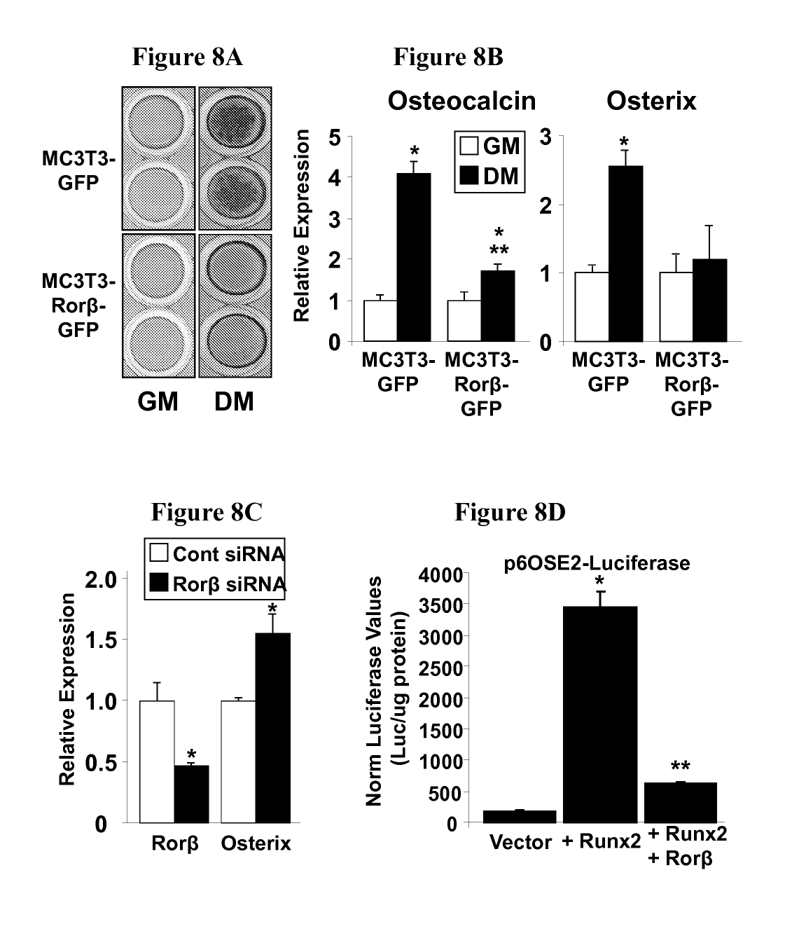

QPCR analysis of these two cell models revealed a 2.5-fold overexpression of Rorβ in MC3T3-Rorβ-GFP cells (FIG. 7B), confirming stable genomic integration and expression of the Rorβ transgene. To determine the amount of Rorβ expression during osteoblastic differentiation in these models, MC3T3-GFP and MC3T3-Rorβ-GFP cells were treated with either growth or osteoblastic differentiation media for 14 days. QPCR analysis demonstrated that Rorβ expression declines 2.5-fold with osteoblastic differentiation medium in the control MC3T3-GFP cell model (FIG. 7C), confirming the data in other models (FIG. 3C and FIG. 6). The identical experiment in the MC3T3-Rorβ-GFP model resulted in increased Rorβ expression levels that did not decline during osteoblast differentiation (FIG. 7C), providing an ideal model to test whether decreased Rorβ expression is prerequisite for osteoblast differentiation in the absence of gross overexpression. Indeed, mineralization capacity of the MC3T3-Rorβ-GFP model following 14 days of differentiation was severely inhibited, as compared to the MC3T3-GFP control model (FIG. 8A). QPCR analysis of two classic bone marker genes, osteocalcin and osterix, revealed significant inhibition of differentiation-induced expression in the MC3T3-Rorβ-GFP model (FIG. 8B). Analysis of other bone marker genes, such as alkaline phosphatase, osteopontin, Runx2, and Col1α1, did not significantly change, demonstrating that Rorβ-dependent inhibition of mineralization may affect a small, but important, subset of bone regulatory genes.

Rorβ was modestly overexpressed about 2.5-fold in the MC3T3-Rorβ-GFP cells over control cells and therefore represents an excellent model to identify differential gene expression with Rorβ overexpression. RNA from both the MC3T3-GFP and MC3T3-Rorβ-GFP cell lines was subjected to microarray analysis using the Mouse WG-6 (version 2.0; Illumina) bead array. Table 4 describes the genes exhibiting altered expression in the MC3T3-Rorβ-GFP cell line (q<0.05 and fold-change >1.5).

TABLE 4
Genes altered in the MC3T3-Rorβ cell line (versus
MC3T3-GFP as control) that fit the criteria of a false
discovery rate (q) <0.05 and fold-change >1.5

| | | Fold-Change(Rorb |
|---|---|---|
| Column ID | qvalue(p-value(Group)) | vs. Ctrl) |
| Aldh3a1 | 1.449E−02 | 3.65499 |
| Ppp1r3c | 1.690E−03 | 3.46533 |
| D0H4S114 | 8.840E−04 | 3.41674 |
| A130047F11Rik | 4.953E−04 | 2.51077 |
| Prelp | 2.609E−03 | 2.39428 |
| Dbp | 7.137E−04 | 2.38212 |
| 9430052C07Rik | 4.586E−04 | 2.3548 |
| D230046H12Rik | 1.156E−02 | 2.30619 |
| Scara5 | 1.397E−02 | 2.25704 |
| Mgp | 4.510E−05 | 2.19755 |
| Aqp1 | 9.662E−04 | 2.1838 |
| Tmem86a | 4.507E−03 | 2.15455 |
| Itga10 | 3.110E−05 | 2.14449 |
| Sulf1 | 1.894E−03 | 2.11863 |
| Spon2 | 7.492E−04 | 2.11862 |
| Trps1 | 5.984E−04 | 2.09913 |
| Sema5a | 3.949E−03 | 2.08027 |
| 2900017F05Rik | 5.773E−03 | 2.07331 |
| Sesn1 | 6.210E−05 | 2.05376 |
| Ank | 3.969E−02 | 2.026 |
| Capn5 | 2.790E−05 | 2.02524 |
| Nfatc4 | 1.390E−05 | 2.02277 |
| Cp | 1.886E−02 | 2.0058 |
| B230343A10Rik | 1.346E−04 | 2.00289 |
| Irx3 | 9.645E−03 | 1.98632 |
| 1110046J11Rik | 1.425E−02 | 1.97808 |
| Igf2bp2 | 5.677E−04 | 1.97176 |
| 5033414K04Rik | 2.591E−02 | 1.96549 |
| Fam122b | 3.567E−03 | 1.95248 |
| 2810410A03Rik | 3.960E−05 | 1.93153 |
| Egr2 | 1.544E−02 | 1.90715 |
| Samd9l | 1.392E−02 | 1.878 |
| Capn6 | 1.390E−05 | 1.87496 |
| Fcgrt | 1.120E−05 | 1.86928 |
| 4732462B05Rik | 4.583E−04 | 1.86008 |
| C130023A14Rik | 1.595E−03 | 1.85603 |
| Zfp36l1 | 2.809E−03 | 1.85513 |
| Gper | 1.277E−04 | 1.85497 |
| BC031353 | 2.052E−04 | 1.84297 |
| Scd2 | 4.065E−04 | 1.83714 |
| Hsd3b7 | 5.187E−03 | 1.83326 |
| Txnip | 3.466E−04 | 1.83276 |
| H2-T23 | 1.966E−03 | 1.83013 |
| Trp53inp1 | 1.578E−02 | 1.8195 |
| 5730593F17Rik | 5.960E−05 | 1.81907 |
| Pgcp | 2.139E−04 | 1.81641 |
| A730017D01Rik | 2.634E−04 | 1.81543 |
| Clip4 | 2.462E−03 | 1.80902 |
| Rab3d | 3.030E−05 | 1.79946 |
| Hist1h1c | 2.155E−03 | 1.79884 |
| 2310047A01Rik | 6.888E−04 | 1.78987 |
| AW049604 | 1.394E−03 | 1.78875 |
| B130038B15Rik | 1.810E−03 | 1.78671 |
| Mme | 2.899E−04 | 1.78524 |
| Gas7 | 1.451E−04 | 1.78206 |
| Ets2 | 2.951E−03 | 1.77984 |
| Adam23 | 3.925E−03 | 1.77769 |
| Rftn2 | 1.709E−04 | 1.77249 |
| 4933421G18Rik | 2.910E−03 | 1.7723 |
| B430110C06Rik | 9.063E−04 | 1.76667 |
| Ppnr | 3.386E−03 | 1.76217 |
| Ahnak2 | 2.235E−02 | 1.75876 |
| Hbp1 | 7.190E−05 | 1.75815 |
| Rdm1 | 4.510E−05 | 1.75776 |
| Nfat5 | 4.392E−03 | 1.75139 |
| Trib2 | 6.413E−04 | 1.74958 |
| Tnnc1 | 5.999E−04 | 1.74364 |
| A730054J21Rik | 1.063E−03 | 1.74272 |
| LOC100048436 | 3.740E−05 | 1.73517 |
| Ypel3 | 4.615E−04 | 1.73478 |
| Phxr4 | 1.160E−02 | 1.72744 |
| Dcn | 2.022E−03 | 1.72407 |
| 4931426K16Rik | 3.761E−03 | 1.72381 |
| Rin2 | 2.634E−04 | 1.71636 |
| Sparcl1 | 5.211E−03 | 1.71488 |
| Sdc2 | 8.350E−05 | 1.71348 |
| Pik3r3 | 8.025E−04 | 1.70884 |
| Adamtsl4 | 5.343E−04 | 1.70789 |
| Tgfb2 | 8.406E−03 | 1.70728 |
| Notch1 | 6.025E−04 | 1.70354 |
| Ptprv | 2.414E−04 | 1.70295 |
| Slc25a12 | 5.677E−04 | 1.70221 |
| Grn | 7.954E−03 | 1.70059 |
| 7330410H16Rik | 2.859E−04 | 1.70041 |
| Wnt10a | 3.682E−03 | 1.69936 |
| LOC381739 | 4.816E−04 | 1.69805 |
| Emp2 | 5.402E−03 | 1.69563 |
| Plekha4 | 6.741E−04 | 1.69507 |
| Pdgfra | 7.110E−05 | 1.688 |
| Rasl11b | 4.271E−02 | 1.68732 |
| Hist1h2bc | 1.279E−03 | 1.68574 |
| Pla2g7 | 1.009E−02 | 1.68133 |
| Grina | 4.623E−02 | 1.68111 |
| Sord | 4.472E−03 | 1.67819 |
| C230066H01Rik | 4.808E−03 | 1.6772 |
| D930007N19Rik | 2.731E−03 | 1.66912 |
| Col18a1 | 6.698E−04 | 1.66176 |
| Iqgap2 | 2.831E−02 | 1.65958 |
| Matn4 | 4.331E−02 | 1.65867 |
| Oasl2 | 3.432E−03 | 1.6554 |
| scl0002975.1_346 | 1.158E−02 | 1.65355 |
| 9030024J15Rik | 4.056E−03 | 1.6523 |
| Ak3 | 2.779E−04 | 1.6513 |
| Ddit4l | 1.595E−02 | 1.63996 |
| Zbtb7c | 3.220E−05 | 1.63746 |
| Tmem2 | 1.646E−03 | 1.63741 |
| Add3 | 3.808E−04 | 1.63701 |
| Adrbk2 | 3.240E−05 | 1.63664 |
| 5830427D02Rik | 6.780E−03 | 1.63544 |
| Lrrc17 | 2.245E−02 | 1.63371 |
| 6430511F03 | 8.240E−07 | 1.63183 |
| Tmem118 | 2.938E−04 | 1.62847 |
| C130085D15Rik | 3.647E−04 | 1.62511 |
| Hist2h2aa2 | 7.701E−03 | 1.62493 |
| Sobp | 1.179E−04 | 1.62055 |
| Fam102a | 2.700E−05 | 1.61667 |
| 6330403M23Rik | 1.438E−02 | 1.61658 |
| AW549877 | 2.395E−03 | 1.6144 |
| B230380D07Rik | 7.988E−04 | 1.60981 |
| Fn1 | 9.407E−04 | 1.60591 |
| Cpe | 3.643E−02 | 1.59996 |
| Pdzrn4 | 2.135E−02 | 1.59969 |
| Pik3ip1 | 5.510E−04 | 1.59945 |
| Brp17 | 4.510E−05 | 1.59873 |
| Appl2 | 4.290E−05 | 1.59619 |
| Arhgap18 | 6.947E−03 | 1.59517 |
| Tsc22d3 | 5.510E−04 | 1.59229 |
| Bcl6 | 8.190E−05 | 1.59098 |
| Tmem100 | 1.494E−02 | 1.58562 |
| 2010005O13Rik | 3.877E−03 | 1.58473 |
| Ghr | 2.337E−03 | 1.58219 |
| Zfp521 | 4.472E−03 | 1.57793 |
| A630082K20Rik | 3.885E−03 | 1.57551 |
| Anpep | 7.937E−04 | 1.57474 |
| Psap | 4.301E−04 | 1.57232 |
| 4631423B10Rik | 5.949E−04 | 1.57069 |
| Insc | 2.087E−02 | 1.56609 |
| D4Ertd681e | 1.067E−03 | 1.56602 |
| Per2 | 1.690E−04 | 1.5642 |
| 2010007H06Rik | 5.761E−03 | 1.564 |
| A130062D16Rik | 2.467E−02 | 1.56329 |
| Iigp2 | 7.937E−04 | 1.56018 |
| Tef | 3.001E−04 | 1.55613 |
| B930008G03Rik | 4.745E−03 | 1.55612 |
| Itgb5 | 1.835E−02 | 1.55366 |
| Ednra | 5.880E−04 | 1.5532 |
| LOC100041388 | 3.932E−02 | 1.55233 |
| F830002E14Rik | 1.379E−03 | 1.55145 |
| Sepp1 | 4.933E−04 | 1.55069 |
| 2300002D11Rik | 3.032E−02 | 1.54666 |
| 4933439C20Rik | 2.731E−04 | 1.54436 |
| Cd9 | 9.502E−04 | 1.54223 |
| Cd82 | 1.298E−03 | 1.54052 |
| 1810011O10Rik | 4.915E−03 | 1.53951 |
| Wnt6 | 1.353E−02 | 1.53397 |
| Prickle1 | 4.541E−02 | 1.53392 |
| 6720422M22Rik | 1.028E−02 | 1.53367 |
| C130092E12 | 6.939E−03 | 1.53364 |
| Dag1 | 7.299E−03 | 1.53036 |
| Serpine2 | 9.128E−03 | 1.52955 |
| Ifi27 | 8.001E−03 | 1.52938 |
| Tcea3 | 4.220E−05 | 1.52723 |
| 1110003O08Rik | 6.918E−04 | 1.52483 |
| Cacna1g | 2.844E−02 | 1.52472 |
| C730013O11Rik | 8.237E−03 | 1.52355 |
| Ugt1a10 | 1.851E−03 | 1.52041 |
| Apobec1 | 2.288E−02 | 1.51941 |
| 2310033F14Rik | 1.120E−05 | 1.51783 |
| Rpl22 | 3.253E−03 | 1.51736 |
| Sox4 | 5.016E−03 | 1.51724 |
| 2700063P19Rik | 2.938E−02 | 1.51578 |
| Cyp4f13 | 1.585E−04 | 1.513 |
| Aldh6a1 | 7.774E−03 | 1.51209 |
| C3 | 2.432E−04 | 1.51173 |
| Scd1 | 8.840E−04 | 1.51127 |
| P2rx4 | 5.962E−03 | 1.51061 |
| Lmcd1 | 3.865E−03 | 1.50918 |
| Rspo3 | 5.272E−03 | 1.50693 |
| 3110078M01Rik | 3.466E−04 | 1.50458 |
| 5330431K02Rik | 1.119E−02 | 1.50404 |
| Tap2 | 6.292E−04 | 1.50234 |
| LOC674135 | 1.020E−02 | 1.50112 |
| Dtx3l | 8.997E−03 | 1.50102 |
| Ufsp1 | 1.920E−04 | −1.50059 |
| Uchl3 | 1.144E−03 | −1.50083 |
| Hist1h4j | 1.396E−03 | −1.50159 |
| Ciapin1 | 9.260E−05 | −1.50339 |
| Pycr2 | 2.490E−05 | −1.50423 |
| Fscn1 | 1.123E−03 | −1.50584 |
| Sgk1 | 4.661E−02 | −1.51112 |
| Gnl3 | 3.525E−04 | −1.5126 |
| Timp1 | 6.022E−03 | −1.51333 |
| Tubb2b | 7.600E−05 | −1.51556 |
| Slc7a6 | 3.057E−03 | −1.51788 |
| Tbrg4 | 1.730E−05 | −1.52118 |
| Bag2 | 1.646E−03 | −1.5226 |
| Plaur | 3.019E−04 | −1.53046 |
| Mak16 | 1.295E−04 | −1.53374 |
| Noc3l | 1.631E−04 | −1.53506 |
| Cirh1a | 8.970E−06 | −1.53921 |
| Lyar | 1.295E−04 | −1.53938 |
| Plekhk1 | 1.506E−02 | −1.54148 |
| Pdss1 | 4.300E−04 | −1.54209 |
| Ppan | 1.063E−03 | −1.54218 |
| Rin1 | 1.779E−03 | −1.54277 |
| Grwd1 | 2.788E−04 | −1.54317 |
| Polr1e | 9.330E−05 | −1.54705 |
| Hsp105 | 4.387E−03 | −1.55056 |
| Gjb3 | 1.323E−03 | −1.55223 |
| Nanos1 | 5.677E−04 | −1.55351 |
| Cited2 | 5.898E−03 | −1.55475 |
| 6330505N24Rik | 9.449E−04 | −1.55824 |
| Mars2 | 3.220E−05 | −1.55852 |
| E130012A19Rik | 1.216E−03 | −1.56165 |
| Rrp1b | 5.272E−03 | −1.56473 |
| 5530400B01Rik | 1.229E−03 | −1.56528 |
| Mthfd2 | 2.822E−02 | −1.56902 |
| Hist1h3d | 3.220E−05 | −1.57002 |
| Pa2g4 | 5.538E−04 | −1.57168 |
| 2210411K11Rik | 2.157E−04 | −1.5727 |
| LOC100044948 | 2.343E−03 | −1.57384 |
| Cox18 | 7.600E−05 | −1.57414 |
| LOC100046898 | 1.118E−03 | −1.57459 |
| Cdr2 | 2.071E−03 | −1.57632 |
| Ebna1bp2 | 9.640E−05 | −1.5792 |
| Mrto4 | 6.972E−04 | −1.58001 |
| Atp1b1 | 1.331E−03 | −1.58021 |
| Schip1 | 1.568E−02 | −1.58513 |
| Myl9 | 1.849E−02 | −1.5869 |
| Hist1h3f | 2.790E−05 | −1.58772 |
| Coq10b | 7.263E−04 | −1.5893 |
| Gdf15 | 4.816E−04 | −1.59078 |
| Nol5a | 7.937E−04 | −1.59104 |
| Ly6a | 1.351E−02 | −1.5925 |
| Hist1h3c | 1.120E−05 | −1.59551 |
| Magohb | 2.651E−03 | −1.59826 |
| Mrps18b | 1.269E−03 | −1.60252 |
| Exosc1 | 4.074E−04 | −1.60259 |
| Hspa5 | 5.644E−03 | −1.60359 |
| Ccne1 | 2.300E−04 | −1.60416 |
| Rasgrp3 | 8.988E−04 | −1.60807 |
| Ctps | 1.729E−03 | −1.609 |
| Snx7 | 1.503E−02 | −1.60999 |
| Uck2 | 1.120E−05 | −1.6122 |
| Rrm2 | 8.141E−04 | −1.61387 |
| Nol1 | 8.090E−05 | −1.61463 |
| Odc1 | 2.312E−04 | −1.61557 |
| Mvk | 1.324E−04 | −1.62678 |
| Scube3 | 2.107E−02 | −1.63429 |
| Hist1h3e | 3.220E−05 | −1.6381 |
| Nus1 | 3.220E−05 | −1.63883 |
| Ddx21 | 1.547E−04 | −1.64281 |
| Inhba | 8.090E−03 | −1.65199 |
| LOC240672 | 1.212E−02 | −1.65233 |
| 1700029F09Rik | 3.001E−04 | −1.65338 |
| Ifrd2 | 1.390E−05 | −1.65659 |
| Nola2 | 2.372E−04 | −1.65897 |
| Eef1e1 | 1.096E−03 | −1.66694 |
| Bxdc1 | 1.295E−04 | −1.66802 |
| Wisp1 | 2.638E−03 | −1.66858 |
| Timm8a1 | 1.580E−05 | −1.67605 |
| Ppa1 | 2.598E−04 | −1.67739 |
| Wisp2 | 3.853E−03 | −1.68207 |
| LOC219106 | 4.065E−04 | −1.68457 |
| Ccrn4l | 2.598E−04 | −1.68884 |
| Tfrc | 4.895E−03 | −1.70605 |
| Cycs | 1.354E−04 | −1.71907 |
| Hist1h3a | 4.510E−05 | −1.71987 |
| Sytl2 | 2.598E−04 | −1.72038 |
| Rras2 | 1.634E−04 | −1.72677 |
| Ccl2 | 3.870E−04 | −1.72979 |
| Ccdc86 | 5.960E−05 | −1.73251 |
| LOC100048295 | 5.187E−03 | −1.73478 |
| Ly6c1 | 7.301E−03 | −1.74479 |
| 5033430l15Rik | 3.940E−03 | −1.75561 |
| 6720458F09Rik | 1.920E−04 | −1.75863 |
| Chac1 | 1.040E−02 | −1.80114 |
| 1110007M04Rik | 1.390E−05 | −1.80675 |
| Dusp8 | 1.182E−03 | −1.81002 |
| Bcat1 | 4.039E−04 | −1.83073 |
| Mical2 | 6.920E−05 | −1.83914 |
| Junb | 1.584E−04 | −1.84681 |
| Rrp9 | 1.350E−05 | −1.852 |
| Id2 | 3.975E−03 | −1.85233 |
| Steap1 | 1.228E−04 | −1.86423 |
| LOC100048332 | 3.326E−02 | −1.87112 |
| Nrn1 | 1.907E−03 | −1.87797 |
| LOC245892 | 5.240E−05 | −1.88537 |
| Gstk1 | 1.390E−05 | −1.88893 |
| Irf5 | 3.691E−03 | −1.89044 |
| Pno1 | 8.970E−06 | −1.89698 |
| Dlk2 | 2.635E−03 | −1.9093 |
| Pkp2 | 2.121E−03 | −1.91012 |
| Srm | 5.984E−04 | −1.91413 |
| Tnfrsf11b | 5.082E−03 | −1.94812 |
| Rrp12 | 1.020E−05 | −1.95697 |
| Car12 | 5.999E−04 | −1.97721 |
| LOC677008 | 8.471E−04 | −1.97758 |
| Dio3 | 6.622E−03 | −1.97884 |
| 6330404C01Rik | 3.436E−02 | −1.9789 |
| Sh2d1b1 | 6.413E−04 | −2.04115 |
| Tnfrsf12a | 1.951E−02 | −2.05581 |
| Gadd45g | 1.040E−02 | −2.05651 |
| Id1 | 1.452E−02 | −2.07059 |
| Sox11 | 6.698E−04 | −2.15487 |
| Siglecg | 1.380E−05 | −2.17391 |
| Npr3 | 4.816E−04 | −2.20059 |
| Dusp1 | 1.917E−04 | −2.25174 |
| Ankrd1 | 3.880E−05 | −2.32056 |
| Ctgf | 2.191E−02 | −2.48403 |
| Actg2 | 4.160E−05 | −2.52786 |
| Ccl7 | 9.640E−05 | −2.56528 |
| Cyr61 | 7.797E−04 | −2.739 |
| Fhl1 | 9.770E−05 | −2.75117 |
| Krt14 | 4.510E−05 | −2.83605 |
| Fos | 1.390E−05 | −3.0572 |
| Rasl11a | 4.527E−03 | −3.24835 |

RNA interference (RNAi) assays using a Rorβ-specific siRNA resulted in a 54% reduction of Rorβ mRNA and concomitantly increased expression of osterix by 55% (FIG. 8C). For siRNA studies, MC3T3-E1 cells were transfected in 24-well plates at a density of 2.6×10⁴ cells/cm² using HyperFect Transfection Reagent (Qiagen) with either a non-specific siRNA control (AllStars Negative Control siRNA) or a mouse-specific Rorβ siRNA (Qiagen) at a concentration of 33 nM according to the manufacturer's protocol. Following incubation at 37° C. for 48 hours, cells were collected, and QPCR analysis was performed as described herein. Contrary to the Rorβ overexpression data (FIG. 8B), osteocalcin expression was not significantly changed with the Rorβ siRNA. Since osterix is a direct transcriptional target of Runx2, it was reasoned that Rorβ may antagonize Runx2 transcriptional activity. To test this possibility, cells were transiently transfected with the Runx2-dependent reporter construct p6OSE2-Luc in the presence of Runx2 and/or Rorβ. U2OS cells were plated in 6-well plates at a density of 2.6×10⁴ cells/cm² the day before transfection. Five-hundred (500) ng of pCMV6-Rorβ (Origene), p6OSE2-Luc, and pCMV-Cbfa1/Runx2 constructs each were transiently transfected (n=6) using X-tremeGENE9 transfection reagent (Roche Diagnostics, Indianapolis, Ind.). Following incubation at 37° C. for 48 hours, cells were harvested in 1× Passive Lysis Buffer, and equal quantities of protein extracts were assayed using Luciferase Assay Reagent on a GloMax® 96 Microplate Luminometer (Promega, Madison, Wis.). Protein concentrations were determined using a BCA Protein Assay Kit (Thermo Scientific, Rockford, Ill.). As expected, Runx2 transfection alone resulted in a 17-fold increase in reporter activity (FIG. 8D). Co-transfection of Rorβ significantly attenuated the ability of Runx2 to activate the reporter, indicating that Rorβ-dependent inhibition of Runx2 function may contribute to the observed inhibition of mineralization in the MC3T3-Rorβ-GFP cell model.

Staple expression of Rorβ in MC3T3-E1 cells inhibited mineralization and expression of osteocalcin and osterix, supporting that suppression of Rorβ may be prerequisite for osteoblastic mineralization to occur. The observation that Rorβ potently inhibits Runx2 transactivation suggests that it may influence bone biology at a fundamental level. Suppression of Rorβ with RNAi resulted in enhanced expression of osterix, consistent with Rorβ-dependent Runx2 antagonism. These findings indicate that Rorβ in bone is a potential target to combat age-related osteoporosis.

### Example 3

### Rorβ Interacts with Runx2

Rorβ represents a pathway important in bone metabolism, and selective repression of this pathway using a small molecule inhibitor may be useful in developing treatments for osteoporosis. Furthermore, the secondary protein structure of Rorβ includes a ligand binding domain (a defining feature of members of the nuclear hormone receptor superfamily), making Rorβ particularly receptive to small molecule interactions. To further define the Rorβ-Runx2 functional interaction, a series of deletions of select domains of Rorβ were produced.

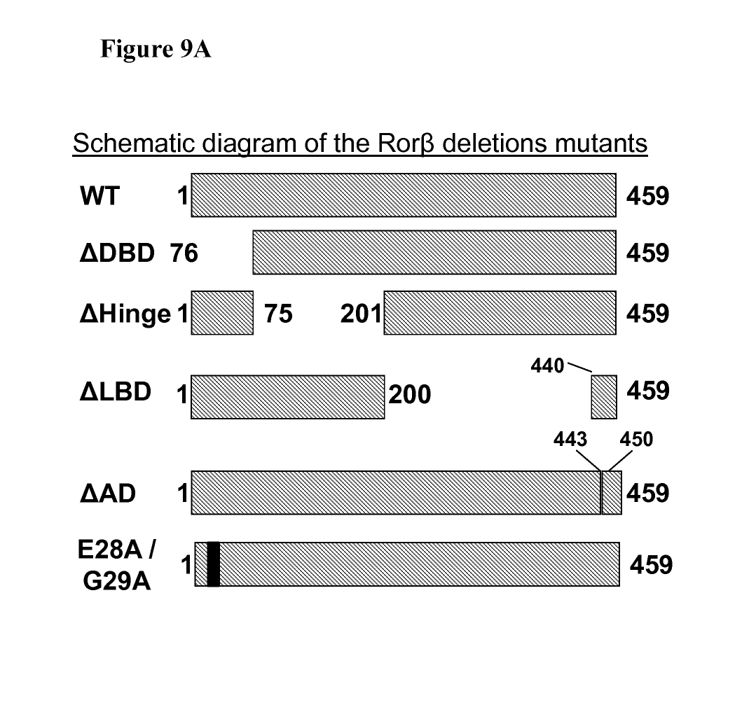

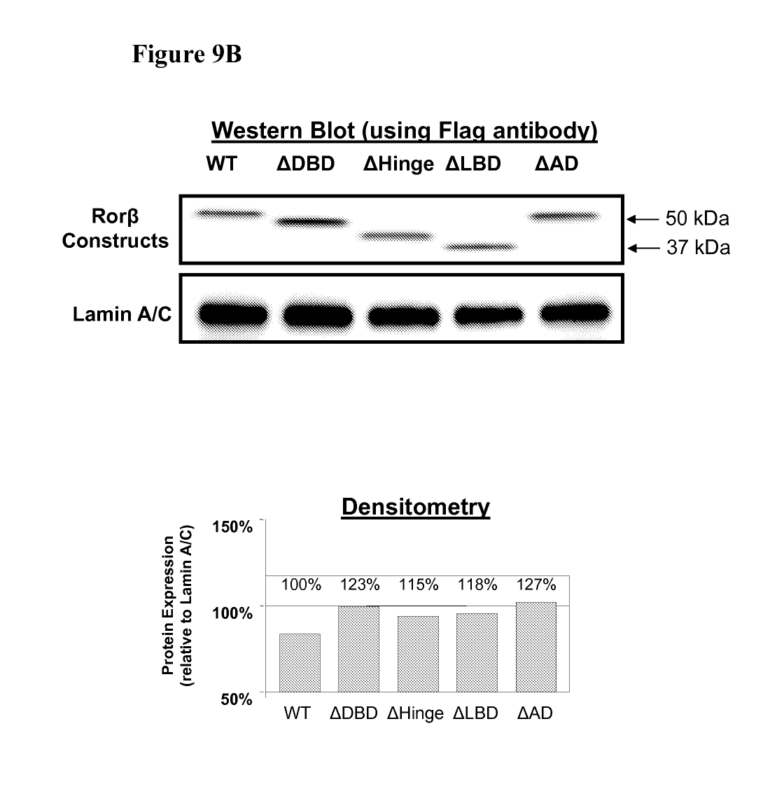

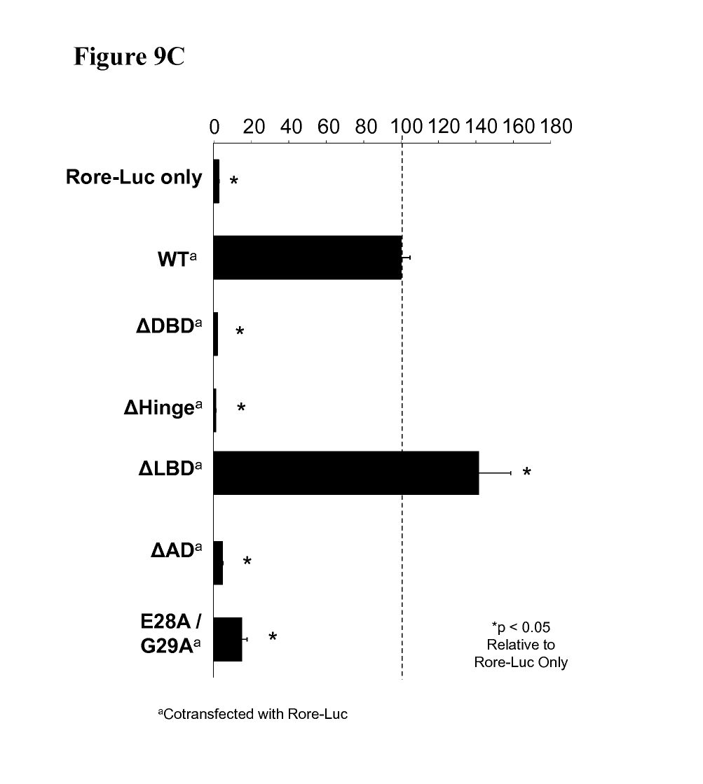

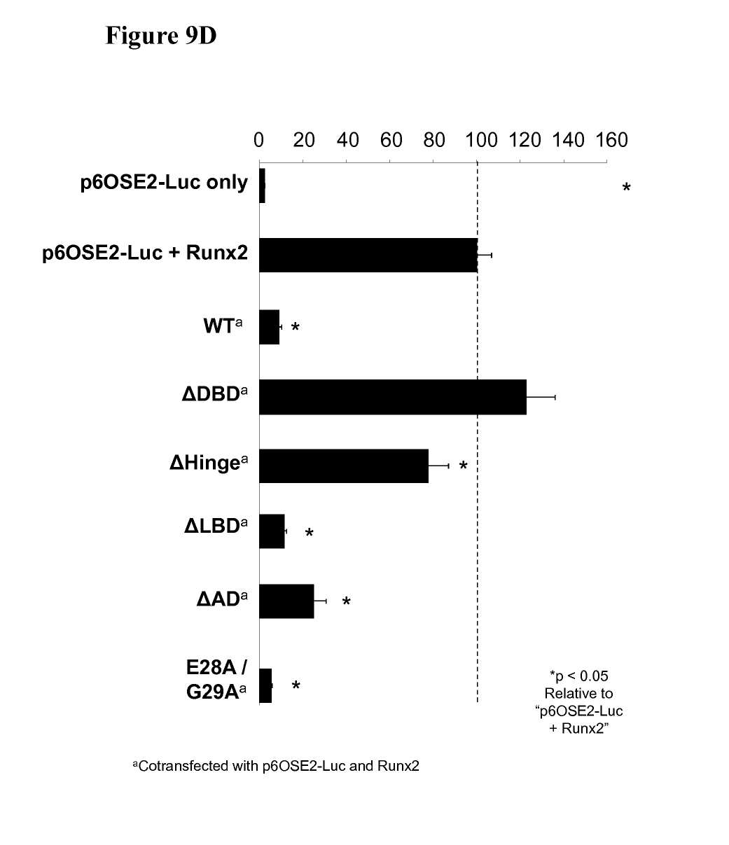

FIG. 9A describes the mutations including deletion of the DNA binding domain (ΔDBD), Hinge (ΔHinge), ligand binding domain (ΔLBD), and the activation domain (ΔAD). It should be noted that all constructs contain a C-terminal FLAG epitope that does not affect Rorβ function. Western blot analysis using an antibody directed against the FLAG epitope demonstrated that the Rorβ mutant polypeptides were expressed at the expected molecular weights (FIG. 9B; top). Furthermore, densitometric analysis using Lamin A/C as a nuclear protein loading control demonstrated similar expression levels among the Rorβ deletion mutants (FIG. 9B; bottom). A double-point mutation in the DBD was produced which suppresses the ability of Rorβ to function through DNA binding directly (E28A/G29A).

The ability of the Rorβ mutants to activate a Rorβ-dependent luciferase reporter construct was tested (Rore-Luc; FIG. 9C). Wild-type (WT) Rorβ activated Rore-Luc 39-fold (over reporter alone), whereas ΔDBD failed to activate the reporter, presumably due to the lack of a DBD. Interestingly, ΔHinge also failed to activate suggesting important sequences are located in this domain for activity. The ΔLBD construct activated the reporter 55-fold, 41% better than WT, suggesting not only that the LBD is dispensable for activation of a Rore, but that the LBD may also possess a negative regulatory domain. The ΔAD construct, which only lacks 7 amino acids (PLYKELF) that are conserved among the Ror family, failed to activate the reporter. The E28A/G29A mutation, designed after the NERKI mutation in estrogen receptor-α which inhibits DNA binding (Jakacka), activated the reporter 6.5-fold less potently than WT Rorβ.

Next, the ability of these Rorβ mutants to suppress Runx2 activation of the p6OSE2-Luc reporter construct was tested (setting the p6OSE2-Luc+Runx2 condition at 100) (FIG. 9D). As described above, WT Rorβ potently suppressed Runx2 activity by 91%. The ΔDBD construct was unable to repress Runx2 activity, and the ΔHinge construct only suppressed the reporter by 28%. The ΔLBD and ΔAD constructs were able to suppress Runx2 activity by 88% and 75%, respectively. These data suggested that the functional interaction between Rorβ and Runx2 is mediated through the DBD and Hinge domains of Rorβ. Interestingly, the E28A/G29A mutation, which poorly activates a Rorβ-Luc reporter construct (FIG. 9C), was able to suppress Runx2 activity by 96%, demonstrating that Runx2 repression by Rorβ is independent of DNA binding. It was interesting that the ΔAD construct, which cannot activate a Rore-Luc reporter, still suppressed Runx2 activity by 75%. This demonstrates that even a transcriptionally incompetent receptor on its cognate element (Rore), still has the ability to repress Runx2, suggesting that these two functions of Rorβ are independent of each other.

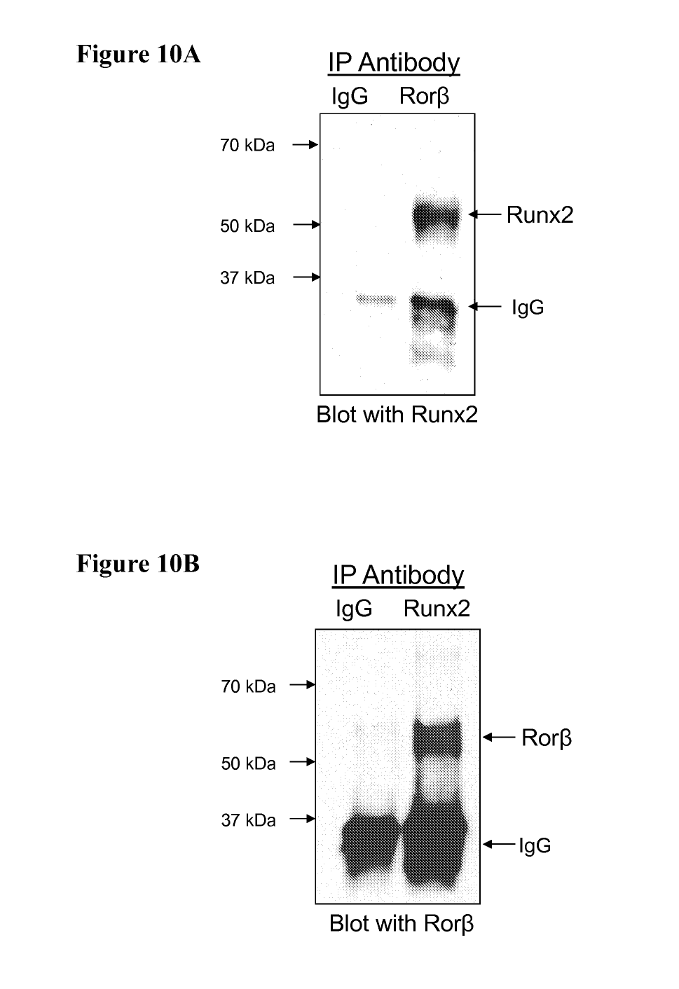

The data in FIG. 9D demonstrates that Rorβ inhibited Runx2 function in a transient transfection analysis, suggesting physical interaction of Rorβ and Runx2. To confirm that these proteins interact, a coimmunoprecipitation assay was performed using specific antibodies that recognize either Rorβ or Runx2. U2OS cells were transiently transfected with Rorβ and Runx2 expression constructs, and nuclear extracts were prepared. An immunoprecipitation using 1 μg of either isotype (IgG), Rorβ, or Runx2 antibodies was performed, and western blotting of the immunoprecipitated protein was performed using the reciprocal antibody. The locations of Runx2, Rorβ, and IgG were indicated by arrows. FIG. 10A reveals that Runx2 protein was detected in Rorβ immunoprecipitates from nuclear extracts of U2OS cells. FIG. 10B demonstrates the reciprocal interaction (Rorβ protein detectable in Runx2 immunoprecipitates). In both experiments, no protein was found in a mock immunoprecipitation using isotype IgG (IgG), demonstrating specificity of the interaction. These data cannot distinguish whether the interaction is direct or via intermediate(s) proteins, however it does establish that Rorβ and Runx2 are present in the same complex(es).

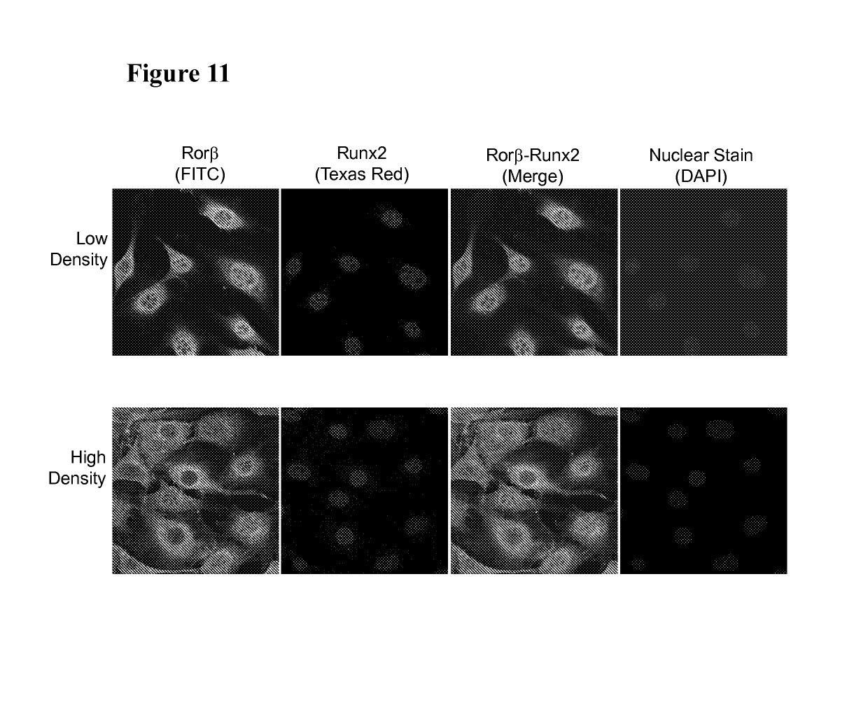

A hypothesis of the function of Rorβ in osteoblasts was formulated whereby Rorβ serves to inhibit Runx2 function prior to the onset of osteoblastic differentiation. Following induction of differentiation, Rorβ levels decline. This allows Runx2 to exert its pro-osteogenic influence. To gain more insight into this interaction in cells, the cellular distribution of Rorβ and Runx2 in low-density and high-density MC3T3-E1 cultures was determined using immunohistochemistry. Rorβ expression was found in both the nucleus and cytoplasm, whereas Runx2 expression was strictly nuclear (FIG. 11). Interestingly, the patterns of Rorβ and Runx2 staining largely overlapped (brown-yellowish color in the Merge panel) in low density cultures. However, at higher density, Rorβ adopted a more perinuclear pattern, and overlap with Runx2 was less apparent. This fits the hypothesis well, since these data suggest that Rorβ and Runx2 are colocalized in low-density cultures where Rorβ inhibits the transcriptional function of Runx2. When the cells grow to higher density, a currently unknown mechanism shifts the cellular distribution of Rorβ away from locations of Runx2 expression. These data also suggest that the Rorβ cellular distribution may be influenced by the stage of the cell cycle.

### Example 4

### Expression of Rorβ in Myoblasts

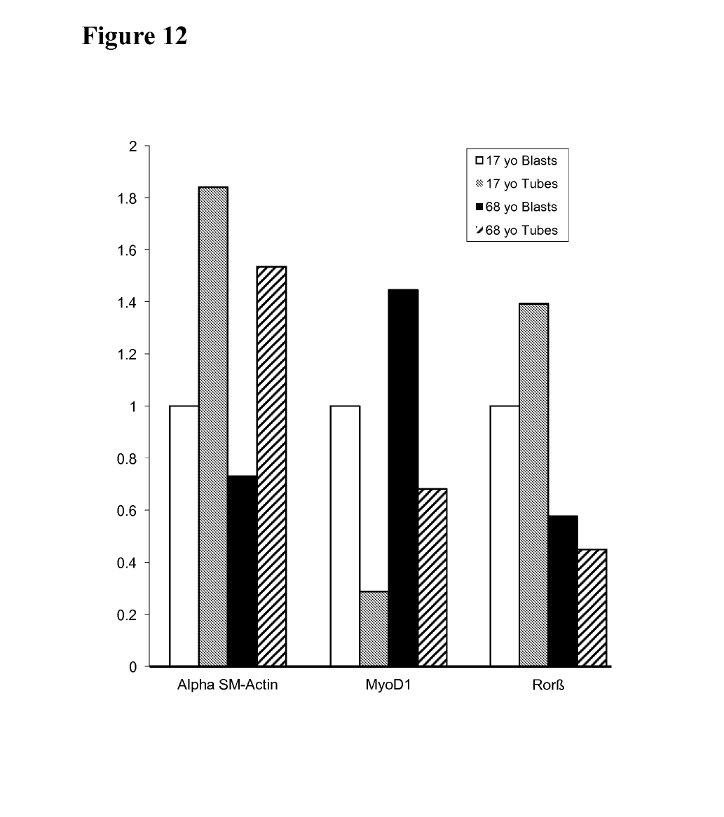

As demonstrated herein, Rorβ levels drastically declined over the course of osteoblastic differentiation and were potently overexpressed in aging osteoblast precursor cells in the bone marrow. These data suggested that Rorβ levels are inversely correlated with osteogenic potential. Therefore, the following was performed to investigate whether Rorβ levels are similarly regulated in differentiating and aged myoblasts as they differentiate into myotubes. QPCR analysis was performed on primary muscle cells isolated from 17—(young) and 68—(old) year old donors that were differentiated into myotubes. Markers for muscle cells (αSM-actin and MyoD1) and Rorβ were assayed using TBP as a QPCR reference gene. αSM-actin was upregulated during muscle cell differentiation, and MyoD1 was repressed (FIG. 12). However, unlike in osteoblasts, Rorβ was not suppressed during myoblast differentiation and was not overexpressed in aging myoblasts/tubes. These results demonstrate the specificity of the observed regulatory patterns in osteoblast and osteoblast precursor cells.

### Example 5

### Effects of Age on Molecular Pathways Regulating Bone Formation in Humans

Bone marrow aspirates and biopsies were used to obtain needle biopsies (1-2 mm diameter) from the posterior iliac crest in 20 young (30±5 years old) and 20 old (73±7 years old) women. QPCR analyses of 288 genes related to bone metabolism, including genes reflecting 17 pre-specified clusters/pathways (e.g., Wnt targets) and 71 genes linked to SNPs from GWAS studies (Estrada et al., Nat. Genet., 44:491-501 (2012)). Genes in pre-specified pathways were analyzed using a cluster analysis (O'Brien Umbrella Test), which tests for concordant changes in multiple genes in the pathway.

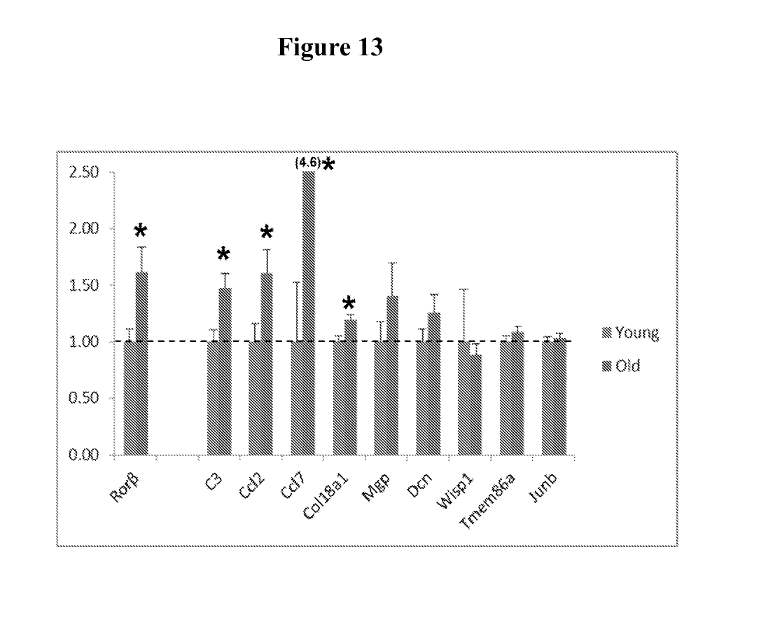

One of the most highly up-regulated pathways in the old women was Notch (P=0.003), which can modulate age-related bone loss in mice (Hilton et al., Nat. Med., 14:306-314 (2008)). Individual significant (P<0.05) gene changes in this pathway were hes1 (1.6×), hey1 (1.8×), heyL (1.5×), and Jag1 (1.2×). In addition, as described herein, Rorβ is an important regulator of osteogenesis that is markedly up-regulated in bone marrow mesenchymal cells from aged versus young mice (see, also, Roforth et al., J. Bone Min. Res., 27:891-901 (2012)). Rorβ itself (1.6×) as well as multiple Rorβ target genes (P=0.001 for the pathway) also were up-regulated in the biopsies from the old women (FIG. 13). Both Notch and Rorβ signaling inhibit runx2 activity, thereby potentially blocking osteoblast differentiation. Interestingly, a panel of stem cell markers was significantly up-regulated with aging (P=0.022), including nestin (2.0×), CD146 (1.4×), and nanog (1.3×), suggesting that activation of Notch and Rorβ signaling may result in a block in osteoblast differentiation with resultant expansion of the stem cell pool within bone.

Of the 71 GWAS genes, 11 were significantly altered with aging, most notably mmp7 (4.0×). Other individual gene changes of interest with aging included rankl (1.6×) and fgf23 (2.0×).

These results demonstrate that coupling needle biopsies of bone to customized QPCR analyses can be used to study genes/pathways regulating bone metabolism in humans. These results also confirm the involvement of Notch and Rorβ signaling with age-related bone loss in humans.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing bone loss within a mammal, wherein said method comprises administering, to said mammal, an inhibitor of a Rorβ polypeptide under conditions wherein the rate of bone loss within said mammal is reduced.

2. The method of claim 1, wherein said mammal is a human.

3. The method of claim 1, wherein said administration is an oral or intravenous administration.

4. The method of claim 1, wherein said inhibitor is an inhibitory anti-Rorβ polypeptide antibody.

5. The method of claim 1, wherein said rate of bone loss is reduced by at least 50 percent.

6. A method for reducing bone loss within a mammal, wherein said method comprises administering, to said mammal, a composition under conditions wherein the rate of bone loss within said mammal is reduced, wherein said composition comprises the ability to reduce Rorβ mRNA expression or Rorβ polypeptide expression.

7. The method of claim 6, wherein said mammal is a human.

8. The method of claim 6, wherein said administration is an oral or intravenous administration.

9. The method of claim 6, wherein said composition comprises a nucleic acid construct having the ability to express a shRNA directed against Rorβ nucleic acid.

10. The method of claim 6, wherein said rate of bone loss is reduced by at least 50 percent.

11. A method for treating osteoporosis, wherein said method comprises administering, to a mammal having osteoporosis, an inhibitor of a Rorβ polypeptide under conditions wherein the rate of bone loss within said mammal is reduced or the bone mass within said mammal is increased.

12. The method of claim 11, wherein said mammal is a human.

13. The method of claim 11, wherein said administration is an oral or intravenous administration.

14. The method of claim 11, wherein said inhibitor is an inhibitory anti-Rorβ polypeptide antibody.

15. The method of claim 11, wherein said rate of bone loss is reduced by at least 50 percent or said bone mass within said mammal is increased by 15 percent.

16. A method for treating osteoporosis, wherein said method comprises administering, to a mammal having osteoporosis, a composition under conditions wherein the rate of bone loss within said mammal is reduced or the bone mass within said mammal is increased, wherein said composition comprises the ability to reduce Rorβ mRNA expression or Rorβ polypeptide expression.

17. The method of claim 16, wherein said mammal is a human.

18. The method of claim 16, wherein said administration is an oral or intravenous administration.

19. The method of claim 16, wherein said composition comprises a nucleic acid construct having the ability to express a shRNA directed against Rorβ nucleic acid.

20. The method of claim 16, wherein said rate of bone loss is reduced by at least 50 percent or said bone mass within said mammal is increased by 15 percent.


SEQUENCE LISTING (144 sequences)

SEQ ID NO: 1 - DNA, 20 bp, Mus musculus
gcccaagatcacctgcactt

SEQ ID NO: 2 - DNA, 20 bp, Mus musculus
atttcctgcgtcgtgttggt

SEQ ID NO: 3 - DNA, 22 bp, Mus musculus
cctatcatgggagacgttgaca

SEQ ID NO: 4 - DNA, 20 bp, Mus musculus
gggtcacctcagcaaaagca

SEQ ID NO: 5 - DNA, 20 bp, Mus musculus
tcaaccaccgcaaacacaac

SEQ ID NO: 6 - DNA, 22 bp, Mus musculus
cagtcaccttcatcagcagctt

SEQ ID NO: 7 - DNA, 22 bp, Mus musculus
agccaagcggaagcttatagag

SEQ ID NO: 8 - DNA, 20 bp, Mus musculus
ggcttgtgcccaattgattt

SEQ ID NO: 9 - DNA, 21 bp, Mus musculus
aaggtggacatgctgcaagag

SEQ ID NO: 10 - DNA, 20 bp, Mus musculus
ctccgtttccggacgtagac

SEQ ID NO: 11 - DNA, 22 bp, Mus musculus
ctgaccttgtgttcacctttgc

SEQ ID NO: 12 - DNA, 21 bp, Mus musculus
ggcctgtttctgtgtcatcca

SEQ ID NO: 13 - DNA, 20 bp, Mus musculus
gaccagatcacgctgctcaa

SEQ ID NO: 14 - DNA, 21 bp, Mus musculus
ccttgtacagatccgcagcat

SEQ ID NO: 15 - DNA, 21 bp, Mus musculus
ggattgtgcacgtgcttaagc

SEQ ID NO: 16 - DNA, 22 bp, Mus musculus
tgggaagaggaaggtgtcatct

SEQ ID NO: 17 - DNA, 20 bp, Mus musculus
gaagccatccaggacaccat

SEQ ID NO: 18 - DNA, 20 bp, Mus musculus
agggtggttgacctgcagat

SEQ ID NO: 19 - DNA, 20 bp, Mus musculus
cccaccaacttcggaatcag

SEQ ID NO: 20 - DNA, 21 bp, Mus musculus
aatgcgagtggtcttccatca

SEQ ID NO: 21 - DNA, 20 bp, Mus musculus
gctccatcgttcgcatcaat

SEQ ID NO: 22 - DNA, 21 bp, Mus musculus
tgccaacggagagacacttct

SEQ ID NO: 23 - DNA, 20 bp, Mus musculus
gcaatcccaagaacgctgat

SEQ ID NO: 24 - DNA, 22 bp, Mus musculus
caatctgtgcggtcactcttca

SEQ ID NO: 25 - DNA, 22 bp, Mus musculus
ctcgagatgctgtcaagtttgg

SEQ ID NO: 26 - DNA, 21 bp, Mus musculus
cggcgtacaagctgtctctct

SEQ ID NO: 27 - DNA, 20 bp, Mus musculus
cgggatccactacggagtca

SEQ ID NO: 28 - DNA, 21 bp, Mus musculus
gctggctcctcctgaagaatc

SEQ ID NO: 29 - DNA, 20 bp, Mus musculus
ctcagcgccctgtgtttttc

SEQ ID NO: 30 - DNA, 20 bp, Mus musculus
tgagaaccagggccgtgtag

SEQ ID NO: 31 - DNA, 20 bp, Mus musculus
aaggcgtccaccattgagat

SEQ ID NO: 32 - DNA, 22 bp, Mus musculus
atgcattctgtctcgtggttgt

SEQ ID NO: 33 - DNA, 20 bp, Mus musculus
cacgcctacgtctccatcaa

SEQ ID NO: 34 - DNA, 20 bp, Mus musculus
tagcatccgtgggaacatca

SEQ ID NO: 35 - DNA, 20 bp, Mus musculus
tgctcacagcgatcgtcatc

SEQ ID NO: 36 - DNA, 20 bp, Mus musculus
caccgcctctctgtccttga

SEQ ID NO: 37 - DNA, 21 bp, Mus musculus
gggactcccaggatttgaaaa

SEQ ID NO: 38 - DNA, 22 bp, Mus musculus
ttttgacgccttctgtaatgca

SEQ ID NO: 39 - DNA, 21 bp, Mus musculus
ggcttccacttcaacgctatg

SEQ ID NO: 40 - DNA, 20 bp, Mus musculus
catgctccgcctgaagaaac

SEQ ID NO: 41 - DNA, 22 bp, Mus musculus
ggaagagcccatcaacgtagag

SEQ ID NO: 42 - DNA, 20 bp, Mus musculus
ccccacatacacggcagatt

SEQ ID NO: 43 - DNA, 22 bp, Mus musculus
agacgaacagtcagcaaaacca

SEQ ID NO: 44 - DNA, 21 bp, Mus musculus
gacctcacaccttccagcaaa

SEQ ID NO: 45 - DNA, 21 bp, Mus musculus
gccgacaatgtgtggtagaca

SEQ ID NO: 46 - DNA, 21 bp, Mus musculus
agcccggaagcacttcttaag

SEQ ID NO: 47 - DNA, 21 bp, Mus musculus
aaaagaagcggtgcaaaatga

SEQ ID NO: 48 - DNA, 22 bp, Mus musculus
gttgctgccctcgtaggtactt

SEQ ID NO: 49 - DNA, 21 bp, Mus musculus
gccatctttgacagggtgcta

SEQ ID NO: 50 - DNA, 21 bp, Mus musculus
ctccgtcttgtccatctgcat

SEQ ID NO: 51 - DNA, 21 bp, Mus musculus
cgcctcactggagacctattg

SEQ ID NO: 52 - DNA, 21 bp, Mus musculus
gtaacagcagcttggcaaacc

SEQ ID NO: 53 - DNA, 21 bp, Mus musculus
gcccgtggagaggattctaga

SEQ ID NO: 54 - DNA, 22 bp, Mus musculus
cgttcatgtcaccgtaggattc

SEQ ID NO: 55 - DNA, 20 bp, Mus musculus
agcgagtcgcacgtagcttt

SEQ ID NO: 56 - DNA, 21 bp, Mus musculus
ttcaggtactcgggcatagga

SEQ ID NO: 57 - DNA, 21 bp, Mus musculus
agtgacctctttggccaacct

SEQ ID NO: 58 - DNA, 21 bp, Mus musculus
ggctgcatttctgaagcatca

SEQ ID NO: 59 - DNA, 22 bp, Mus musculus
cccaagtatccccatgaagtga

SEQ ID NO: 60 - DNA, 21 bp, Mus musculus
agagaagcctggcagctgatt

SEQ ID NO: 61 - DNA, 20 bp, Mus musculus
catgggccaccactttatgg

SEQ ID NO: 62 - DNA, 22 bp, Mus musculus
gtcctctggctccagtttagca

SEQ ID NO: 63 - DNA, 20 bp, Mus musculus
cggttcagcgaggaagaatg

SEQ ID NO: 64 - DNA, 21 bp, Mus musculus
ccccgtttgtgagtgcatact

SEQ ID NO: 65 - DNA, 22 bp, Mus musculus
gtttttcgtccgtttggtaggt

SEQ ID NO: 66 - DNA, 22 bp, Mus musculus
tgctgccggacagtaacatatc

SEQ ID NO: 67 - DNA, 20 bp, Mus musculus
aggtggatgctgcggagtac

SEQ ID NO: 68 - DNA, 20 bp, Mus musculus
agaaaggccacaggcatcag

SEQ ID NO: 69 - DNA, 20 bp, Mus musculus
atgatgaaaggcggcatacg

SEQ ID NO: 70 - DNA, 22 bp, Mus musculus
tctgacgcttgtgcttcaacat

SEQ ID NO: 71 - DNA, 22 bp, Mus musculus
catcagtaacaagggcatggaa

SEQ ID NO: 72 - DNA, 20 bp, Mus musculus
gtcgtacaccgggaccacat

SEQ ID NO: 73 - DNA, 20 bp, Mus musculus
tacggtgtggcatcctgtga

SEQ ID NO: 74 - DNA, 20 bp, Mus musculus
ctcccctggatggtcctctt

SEQ ID NO: 75 - DNA, 21 bp, Mus musculus
tttccccacctgctaaaaagc

SEQ ID NO: 76 - DNA, 22 bp, Mus musculus
cttgtcctgctcaacccctagt

SEQ ID NO: 77 - DNA, 21 bp, Mus musculus
gatccccagaccaagtgtgaa

SEQ ID NO: 78 - DNA, 21 bp, Mus musculus
tcgccacacactaagcacagt

SEQ ID NO: 79 - DNA, 21 bp, Mus musculus
cggtggcagtgtgaaattgta

SEQ ID NO: 80 - DNA, 22 bp, Mus musculus
ctccaaatcctgcaagatgtca

SEQ ID NO: 81 - DNA, 22 bp, Mus musculus
gctcccccagtgttgaaaatag

SEQ ID NO: 82 - DNA, 22 bp, Mus musculus
cttgaaagaggagagcccacat

SEQ ID NO: 83 - DNA, 22 bp, Mus musculus
ctgtcactatggcgtgcttacc

SEQ ID NO: 84 - DNA, 20 bp, Mus musculus
ttatgctgcccttccattgc

SEQ ID NO: 85 - DNA, 22 bp, Mus musculus
tgacaacaaccaaccagattcc

SEQ ID NO: 86 - DNA, 21 bp, Mus musculus
gcctctctccaagctcattga

SEQ ID NO: 87 - DNA, 20 bp, Mus musculus
gtgttgatgttcccgccttt

SEQ ID NO: 88 - DNA, 20 bp, Mus musculus
cccgtgtcgatcagtgatga

SEQ ID NO: 89 - DNA, 21 bp, Mus musculus
gcgcttagcatacaggtccaa

SEQ ID NO: 90 - DNA, 21 bp, Mus musculus
gaccaccccattgcaaaagat

SEQ ID NO: 91 - DNA, 22 bp, Mus musculus
cagtgtcgggatggttaaggaa

SEQ ID NO: 92 - DNA, 21 bp, Mus musculus
cagacgacctctcctcccttt

SEQ ID NO: 93 - DNA, 20 bp, Mus musculus
ctgtgcgtgctgatcgaatg

SEQ ID NO: 94 - DNA, 21 bp, Mus musculus
gcccggtctctcttgtacatg

SEQ ID NO: 95 - DNA, 21 bp, Mus musculus
tctgcaccagggtcagagact

SEQ ID NO: 96 - DNA, 21 bp, Mus musculus
acgtttttcccggagttgttc

SEQ ID NO: 97 - DNA, 21 bp, Mus musculus
tcaagaggagcatttgcaaca

SEQ ID NO: 98 - DNA, 21 bp, Mus musculus
tccgggacatgacacagttct

SEQ ID NO: 99 - DNA, 20 bp, Mus musculus
cacagattcccaaagcacct

SEQ ID NO: 100 - DNA, 20 bp, Mus musculus
gggatggaggagagaaggtc

SEQ ID NO: 101 - DNA, 22 bp, Mus musculus
cctgagtctgacaaagccttca

SEQ ID NO: 102 - DNA, 22 bp, Mus musculus
gccggagtctgttcactacctt

SEQ ID NO: 103 - DNA, 22 bp, Mus musculus
gcttcacctacagcacccttgt

SEQ ID NO: 104 - DNA, 21 bp, Mus musculus
tgactgtcttgccccaagttc

SEQ ID NO: 105 - DNA, 22 bp, Mus musculus
cccggtgaaagtgactgattct

SEQ ID NO: 106 - DNA, 21 bp, Mus musculus
gatctgggtgcaggctgtaaa

SEQ ID NO: 107 - DNA, 20 bp, Mus musculus
gaggaggtggtggctgacaa

SEQ ID NO: 108 - DNA, 20 bp, Mus musculus
caccttgccatgtttgcaat

SEQ ID NO: 109 - DNA, 22 bp, Mus musculus
ggcacagacagaagcttgatga

SEQ ID NO: 110 - DNA, 21 bp, Mus musculus
gaatgcgccctaaatcactga

SEQ ID NO: 111 - DNA, 20 bp, Mus musculus
ggaggtttcactccattcca

SEQ ID NO: 112 - DNA, 20 bp, Mus musculus
tagaaggagcaggggacaga

SEQ ID NO: 113 - DNA, 21 bp, Mus musculus
tctcaggaatcgccaactttg

SEQ ID NO: 114 - DNA, 22 bp, Mus musculus
cgcgggactgtagtagtcagaa

SEQ ID NO: 115 - DNA, 22 bp, Mus musculus
gggacgacacagatcaacaaaa

SEQ ID NO: 116 - DNA, 21 bp, Mus musculus
ccctttccgttgaacctgatt

SEQ ID NO: 117 - DNA, 20 bp, Mus musculus
tgctctcccagttgccagat

SEQ ID NO: 118 - DNA, 22 bp, Mus musculus
aatcacccgtcctctcttcaga

SEQ ID NO: 119 - DNA, 20 bp, Mus musculus
tctccacggcaacatttttg

SEQ ID NO: 120 - DNA, 20 bp, Mus musculus
ctggcggaacttgcaattct

SEQ ID NO: 121 - DNA, 22 bp, Mus musculus
tgctgccctcctcagaaatatc

SEQ ID NO: 122 - DNA, 20 bp, Mus musculus
gttcggccccagtcactaga

SEQ ID NO: 123 - DNA, 22 bp, Mus musculus
ccttggctgagacacaatgttg

SEQ ID NO: 124 - DNA, 21 bp, Mus musculus
gccttcggctgactgatttct

SEQ ID NO: 125 - DNA, 20 bp, Mus musculus
acttgtgcacgctgccttct

SEQ ID NO: 126 - DNA, 21 bp, Mus musculus
tgacctctgtggcatcattcc

SEQ ID NO: 127 - DNA, 21 bp, Mus musculus
agtccctgccctttgtacaca

SEQ ID NO: 128 - DNA, 22 bp, Mus musculus
ggcctcactaaaccatccaatc

SEQ ID NO: 129 - DNA, 21 bp, Mus musculus
cactgaccggcctgtatgcta

SEQ ID NO: 130 - DNA, 22 bp, Mus musculus
tgggtggcgtgagtatacttga

SEQ ID NO: 131 - DNA, 20 bp, Mus musculus
gtggtccctgctgctctcaa

SEQ ID NO: 132 - DNA, 22 bp, Mus musculus
ccccaggtaagcaaactttctg

SEQ ID NO: 133 - DNA, 22 bp, Mus musculus
gccttacggcacaggacttact

SEQ ID NO: 134 - DNA, 22 bp, Mus musculus
gctgtctttgttgctcttccaa

SEQ ID NO: 135 - DNA, 20 bp, Mus musculus
gggaagcccatcaccatctt

SEQ ID NO: 136 - DNA, 20 bp, Mus musculus
gcctcaccccatttgatgtt

SEQ ID NO: 137 - DNA, 21 bp, Mus musculus
ggaggagttctttgcccgtaa

SEQ ID NO: 138 - DNA, 22 bp, Mus musculus
gtgcttataggaggctgcatca

SEQ ID NO: 139 - DNA, 20 bp, Mus musculus
cgaattgacttgcgtttcca

SEQ ID NO: 140 - DNA, 22 bp, Mus musculus
atgtgccgttccaccttatagc

SEQ ID NO: 141 - DNA, 22 bp, Mus musculus
ggttcccaaagatgtcaatgct

SEQ ID NO: 142 - DNA, 22 bp, Mus musculus
caaactggatggtacgcttggt

SEQ ID NO: 143 - DNA, 22 bp, Mus musculus
cgtgattagcgatgatgaacca

SEQ ID NO: 144 - DNA, 21 bp, Mus musculus
tccaaatcctcggcataatga